(19) United States
(12) Patent Application Publication
(10) Pub. No.: US 2026/0092314 A1
(43) Pub. Date: Apr. 2, 2026

(54) RNA-FACS FOR RARE CELL ISOLATION AND DETECTION OF GENETIC VARIANTS

(71) Applicant: Myriad Women's Health, Inc., South San Francisco, CA (US)

(72) Inventors: Dale Muzzey, South San Francisco, CA (US); Matthew LaBella, South San Francisco, CA (US); Ravi Patel, South San Francisco, CA (US)

(73) Assignee: Myriad Women's Health, Inc., South San Francisco, CA (US)

(21) Appl. No.: 19/112,211

(22) PCT Filed: Jul. 3, 2023
(86) PCT No.: PCT/US2023/026851

§ 371 Date: Mar. 14, 2025

Related U.S. Application Data
(60) Provisional application No. 63/407,582, filed on Sep. 16, 2022.

(51) Int. Cl. C12Q 1/6827; C12Q 1/6809; C12Q 1/6869; C12Q 1/6883

(52) U.S. Cl. CPC C12Q 1/6827; C12Q 1/6809; C12Q 1/6869; C12Q 1/6883; C12Q 2600/118; C12Q 2600/156

(57) ABSTRACT

The present disclosure provides technologies for efficient, sensitive, and/or accurate separation and/or isolation of subpopulations of cells, including rare cells, and methods of detecting of the presence or absence of genetic variants in isolated subpopulations of cells.

### CROSS REFERENCE TO RELATED APPLICATION

This application claims the benefit of and priority to U.S. Provisional Ser. No. 63/407,582 , filed on Sep. 16, 2022, the contents of which are incorporated herein by reference in its entirety.

### BACKGROUND

The following description of the background of the present technology is provided simply as an aid in understanding the present technology and is not admitted to describe or constitute prior art to the present technology.

Current technologies for separating (e.g., isolating) cells types can generally be divided into techniques that require the cells of interest to be differentially tagged (e.g., with antibodies directed to cell type-specific cell surface polypeptides) and techniques that separate cell types using differences in the physical properties of the cells (e.g., cell size, cell shape, dielectrophoretic mobility). Such technologies can be limited due to, for example, overlapping physical properties of varying cell types, the small subset of polypeptides expressed on cell surfaces that can be used to differentiate cell types, and the expense and/or availability of antibodies directed to cell type-specific cell surface polypeptides.

Efficient and accurate separation and/or isolation of subpopulations of cells, including rare cells, from samples (e.g., fluid samples, including, for example, whole blood) is useful in many clinical and research applications. For example, some diagnostic and detection assays require a step of isolating cells from a sample (e.g., whole blood) to avoid interference of other elements in the sample. Diagnostic and detection assays (e.g., DNA sequencing, RNA sequencing) can be further complicated by the harsh conditions (e.g., cross-linking, fixation) that can be required for isolation of subpopulations of cells from samples.

Accordingly, there remains a need for technologies for effective cell sorting (e. g, isolation, and in particular, isolation of rare cells) and subsequent diagnostic or detection assays, including, for example, detection of genetic variants.

### SUMMARY OF THE INVENTION

The present disclosure provides, among other things, technologies for isolating cells and detecting the presence or absence of a genetic variant in isolated cells (e.g., rare cells).

In one aspect, the present disclosure provides a method of genotyping a rare cell comprising: (a) contacting a sample with one or more nucleic acid probes comprising a nucleic acid sequence complementary to one or more rare cell-specific transcripts, wherein the nucleic acid probe comprises a detectable marker, thereby detectably labeling the rare cells; (b) separating and collecting the detectably labeled rare cell from one or more undesired sample components, thereby isolating the rare cell; and (c) genotyping the isolated rare cell.

In some embodiments, the rare cell is a circulating fetal cell (CFC). In some embodiments, the one or more rare-cell specific transcripts is a fetal cell-specific transcript.

In some embodiments, the methods may further comprise diagnosing a fetus with a disease and/or disorder or determining that a fetus is at an increased risk of having a disease and/or disorder based on the presence or absence of a genetic variant in the circulating fetal cells.

In some embodiments, the one or more rare cell-specific transcripts are patient-specific.

In some embodiments, the rare cell is present in the sample at an abundance of about 1e⁻⁴% of the total number of cells in the sample.

In some embodiments, the sample comprises maternal blood. In some embodiments, the sample comprises placental cells.

In some embodiments, the one or more nucleic acid probes comprise hybridization chain reaction probes.

In some embodiments, the detectable marker is or comprises a fluorescent molecule.

In some embodiments, the detectably labeled rare cells are separated from one or more undesired sample components using Fluorescence Activated Cell Sorting (FACS).

In some embodiments, the one or more rare cell-specific transcript comprises CSH-1, IGHG4, CSH2, MIR4280HG, or any combination thereof.

In some embodiments, methods of the present disclosure further comprise verifying the genetic identity of the isolated rare cell.

In some embodiments, methods of the present disclosure further comprise sequencing at least one nucleic acid from the isolated rare cell.

In some embodiments, genotyping may comprise one or more of karyotyping, polymerase chain reaction (PCR), short tandem repeat (STR) profiling, single nucleotide polymorphism (SNP) genotyping, DNA sequencing, RNA sequencing, use of cell type-specific nucleic acid probes, or any combination thereof.

In one aspect, the present disclosure provides a method of detecting the presence or absence of a genetic variant in a rare cell comprising: (a) contacting a sample with one or more nucleic acid probes comprising a nucleic acid sequence complementary to one or more rare cell-specific transcripts, wherein the nucleic acid probe comprises a detectable marker, thereby detectably labeling a rare cell; (b) separating the detectably labeled rare cell from one or more undesired sample components, thereby enriching the rare cell; (c) sequencing at least one nucleic acid from the rare cell, to obtain sequence reads; and (d) detecting the presence or absence of the genetic variant based on the sequencing reads.

In some embodiments, the rare cells comprise circulating fetal cells.

In some embodiments, methods of the present disclosure further comprise diagnosing a fetus with a disease and/or disorder based on the presence or absence of a genetic variant in the circulating fetal cells. In some embodiments, the methods of the present disclosure further comprise diagnosing a fetus with a disease and/or disorder or determining that a fetus is at an increased risk of having a disease and/or disorder based on the presence or absence of a genetic variant in the circulating fetal cells.

In some embodiments, the rare cells are present in the sample at an abundance of about 1 rare cell in 1 million cells in the sample. In some embodiments, the rare cells are present in the sample at an abundance of about 1e⁻⁴% of the total number of cells in the sample.

In some embodiments, the sample comprises maternal blood. In some embodiments, the sample comprises placental cells.

In some embodiments, the one or more nucleic acid probes comprise hybridization chain reaction probes.

In some embodiments, the detectable marker is or comprises a fluorescent molecule.

In some embodiments, the detectably labeled rare cells are separated from one or more undesirable sample components using Fluorescence Activated Cell Sorting (FACS).

In some embodiments, sequencing at least one nucleic acid from the enriched population of rare cells comprises next generation sequencing.

The following detailed description is exemplary and explanatory, but it is not intended to be limiting.

### BRIEF DESCRIPTION OF THE DRAWINGS

FIG. 1 shows detection/isolation of diluted cells using a HCR initiator probe set that specifically hybridizes to 18S ribosomal RNA and an amplifier probe comprising an Alexa Fluor 488 detectable marker followed by FACS.

FIG. 2 demonstrates background fluorescence by flow cytometery, comparing fixed cells not treated in the HCR protocol to the negative controls that did run through the HCR protocol. Negative controls show elevated 488 excitation/˜520 emission intensity, due to increased autofluorescence from the buffers in the HCR protocol. Controls include: autofluorescence (AF, no probe, no amplifier), non-specific detection (NSD, GFP(−) probe+Alexa488 amplifier), non-specific amplification (NSA, no probe+amplifier).

FIG. 3 shows the limit of detection (LOD) experimental scheme. The flow data on the left shows gating parameter based on Alexa488 emission intensity (negative control on top, undiluted 18S labeled cells on bottom). The table describes the dilution series of cells labeled using the HCR initiator probe set that specifically hybridizes to 18S ribosomal RNA and an amplifier probe comprising an Alexa Fluor 488 detectable marker followed by FACS.

FIG. 4 demonstrates level of detection of diluted cells using a HCR initiator probe set that specifically hybridizes to 18S ribosomal RNA and an amplifier probe comprising an Alexa Fluor 488 detectable marker followed by FACS.

FIG. 5 shows detection/isolation of diluted cells using a HCR initiator probe set that specifically hybridizes to 18S ribosomal RNA and an amplifier probe comprising an Alexa Fluor 647 detectable marker followed by FACS.

FIG. 6 demonstrates background fluorescence by flow cytometery, comparing fixed cells not treated in the HCR protocol to the negative controls that did run through the HCR protocol. Negative controls show elevated 647 excitation/˜720 emission intensity, due to increased autofluorescence from the buffers in the HCR protocol. Controls include: autofluorescence (AF, no probe, no amplifier), non-specific detection (NSD, GFP(−) probe+Alexa647 amplifier), non-specific amplification (NSA, no probe+amplifier).

FIG. 7 demonstrates use of a HCR amplifier probe comprising an Alexa Fluor 647 detectable marker has lower background than that of a HCR amplifier probe comprising an Alexa Fluor 488 detectable marker.

FIG. 8 demonstrates level of detection of diluted cells using a HCR initiator probe set that specifically hybridizes to 18S ribosomal RNA and an amplifier probe comprising an Alexa Fluor 647 detectable marker followed by FACS.

FIG. 9 demonstrates level of detection of diluted cells using a HCR initiator probe set that specifically hybridizes to 18S ribosomal RNA and an amplifier probe comprising an Alexa Fluor 647 detectable marker followed by FACS.

FIG. 10 shows gating to remove dead cells and cellular debris by flow cytometry.

FIGS. 11A-11B shows an exemplary work flow for the detection/isolation of Circulating Fetal Cells (CFCs) using Y-chromosome-specific nucleic acid probes.

FIG. 12 shows an exemplary work flow for the detection/isolation of CFCs using Y-chromosome-specific nucleic acid probes.

FIGS. 13A-13C demonstrates detection/isolation of male verse female CFCs using Y-chromosome-specific nucleic acid probes followed by FACS. 13A pooled Y-chromosome nucleic acid probes. 13B UTY-specific nucleic acid probes. 13C RPS4Y1-specific nucleic acid probes.

FIG. 14 shows an exemplary workflow for the detection/isolation of CFCs using fetal/placental-specific nucleic acid probes and subsequent detection of the presence or absence of a genetic variant.

FIGS. 15A-15B demonstrates detection/isolation putative CFCs using pooled fetal/placental cell-specific nucleic acid probes (including probes that specifically hybridize CSH-1/2, IGHG4, and MIR4280HG) and FACS. 15A Negative control group. 15B Pooled fetal/placental-specific nucleic acid probe.

FIGS. 16A-16B demonstrates detection/isolation of putative CFCs using IGHG4-specific nucleic acid probes and FACS. 16A Negative control group. 16B IGHG4-specific nucleic acid probes.

FIGS. 17A-17B demonstrates detection/isolation of putative CFCs using MIR4280HG-specific nucleic acid probes and FACS. 17A Negative control group. 17B MIR4280HG specific nucleic acid probes.

FIGS. 18A-18B demonstrates an additional negative control for detection/isolation of putative CFCs using PAEP-specific nucleic acid probes and FACS. 18A Negative control group. 18B PAEP-specific nucleic acid probes.

FIG. 19 shows an exemplary workflow for the detection/isolation of putative CFCs using fetal/placental-specific nucleic acid probes comprising a CSH-1/2-specific nucleic acid probe and subsequent detection of the presence or absence of a genetic variant.

FIGS. 20A-20B demonstrates detection/isolation of putative CFCs using 64 nM of pooled fetal/placental cell-specific nucleic acid probes comprising a CSH-1/2-specific nucleic acid probe and FACS. 20A Negative control group. 20B Pooled fetal/placental-specific nucleic acid probe.

FIGS. 21A-21B demonstrates detection/isolation of putative CFCs using 16 nM of pooled fetal/placental cell-specific nucleic acid probes comprising a CSH-1/2-specific nucleic acid probe and FACS. 21A Negative control group. 21B Pooled fetal/placental-specific nucleic acid probe.

FIGS. 22A-22B demonstrates detection/isolation of putative CFCs using CSH-1/2-specific nucleic acid probes and FACS. 22A Negative control group. 22B CSH-1/2-specific nucleic acid probes.

FIG. 23 shows an exemplary workflow for characterization of isolated cells using whole genome amplification and Next Generation Sequencing (NGS).

FIG. 24 shows an exemplary workflow for characterization of isolated cells using direct amplification of dbSNP sites.

FIG. 25 shows an exemplary computational pipeline.

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No.4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Efficient and accurate separation and/or isolation of subpopulations of cells, including rare cells, from samples (e.g., fluid samples, including, for example, whole blood) is useful in many clinical and research applications. Diagnostic and detection assays (e.g., DNA sequencing, RNA sequencing) can be further complicated by the harsh conditions (e.g., cross-linking, fixation) that can be required for separation and/or isolation of subpopulations of cells from samples. In some embodiments, the present disclosure provides, among other things, technologies for isolation of subpopulations of cells (e.g., rare cells) and methods of detecting of the presence or absence of a genetic variant in cells (e.g., isolated rare cells) that are highly sensitive or that may provide improved sensitivity over currently utilize cfDNA-based non-invasive prenatal screening (NIPS). In some embodiments, the present disclosure provides methods of isolating cells (e.g., rare cells) comprising contacting a sample with one or more nucleic acid probes complementary to one or more cell type-specific transcripts and separating the cells from one or more undesired sample components. In some embodiments, the present disclosure provides technologies for detecting the presence or absence of a genetic variant in cells (e.g., rare cells) comprising contacting a sample with one or more nucleic acid probes complementary to one or more cell type-specific transcripts, separating the cells from one or more undesired sample components, thereby enriching the population of cells, sequencing the enriched population of cells, and detecting the presence or absence of a genetic variant based on the sequencing reads.

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value. Further, when a value is modified by “about,” it should be understood that such a disclosure encompasses both the stated value and, independently, a range associated with that value. For example, “about” may encompass plus or minus 10%, such that the phrase “about 10” should be understood as both “10” and “a range of 9 to 11.”

The term “comparable” is used herein to describe two (or more) sets of conditions, circumstances, individuals, or populations that are sufficiently similar to one another to permit comparison of results obtained or phenomena observed. In some embodiments, comparable sets of conditions, circumstances, individuals, or populations are characterized by a plurality of substantially identical features and one or a small number of varied features. Those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied. Those skilled in the art will appreciate that relative language used herein (e.g., enhanced, activated, reduced, inhibited, etc.) will typically refer to comparisons made under comparable conditions.

The terms “complementary” or “complementarity,” as used herein, refer to the natural binding of polynucleotides under permissive salt and temperature conditions by base-pairing. For the sequence A-G-T, the complementary sequence is T-C-A, the reverse complement is A-C-T and the reverse sequence is T-G-A. Complementarity between two single stranded molecules may be partial, in which only some of the nucleic acids bind, or it may be complete when total complementarity exists between the single stranded molecules. The degree of complementarity between nucleic acid strands has significant effects on the efficiency and strength of hybridization between nucleic acid strands.

A device or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

As used herein, “diagnostic information” or “information for use in diagnosis” is any information that is useful in determining whether a patient has a disease, disorder, and/or condition and/or in classifying the disease, disorder, and/or condition into a phenotypic category or any category having significance with regard to prognosis of the disease or condition, or likely response to treatment (either treatment in general or any particular treatment) of the disease or condition. Similarly, diagnosis refers to providing any type of diagnostic information, including, but not limited to, whether a subject is likely (e.g., at an increased or high risk) to have a disease or condition, state, staging or characteristic of the disease or condition as manifested in the subject, information related to prognosis and/or information useful in selecting an appropriate treatment. Selection of treatment may include the choice of a particular therapeutic agent or other treatment modality such as surgery, etc., a choice about whether to withhold or deliver therapy, a choice relating to dosing regimen (e.g., frequency or level of one or more doses of a particular therapeutic agent or combination of therapeutic agents), etc.

As used herein, “genotyping” refers to genetic assessment, analysis, characterization, or quantification of one or more genetic features of one or more cells. Genotyping can comprise, for example, karyotyping, DNA-based methods (e.g., polymerase chain reaction (PCR), short tandem repeat (STR) profiling, single nucleotide polymorphism (SNP) genotyping, DNA sequencing), RNA-based methods (e.g., RNA sequencing), and use of cell type-specific nucleic acid probes (e.g., variant-specific nucleic acid probes).

As used herein, “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

As used herein, “nucleic acid” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxy cytidine). In some embodiments, a nucleic acid is, comprises, or consists of one or more nucleoside analogs (e.g., 2-aminoadenosine, 2-thiothymidine, inosine, pyrrolo-pyrimidine, 3 methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereol). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity. In some embodiments, a nucleic acid is conjugated to a detectable marker (e.g., a fluorophore). Those skilled in the art, reading the present specification, will appreciate that ligation oligonucleotide sets, activating nucleic acids, and/or guide RNAs can each be engineered and/or manipulated, e.g., to incorporate nucleotide analogs, etc.

As used herein, the terms “prognostic information” and “predictive information” are used interchangeably to refer to any information that may be used to indicate any aspect of the course of a disease, disorder, and/or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

As used herein, “reference”, as those of skill in the art will appreciate, in many embodiments described herein, is a determined value or characteristic of interest is compared with an appropriate reference. In some embodiments, a reference value or characteristic is one determined for a comparable cohort, individual, population, or sample. In some embodiments, a reference value or characteristic is tested and/or determined substantially simultaneously with the testing or determination of the characteristic or value of interest. In some embodiments, a reference characteristic or value is or comprises a historical reference, optionally embodied in a tangible medium. Typically, as would be understood by those skilled in the art, a reference value or characteristic is determined under conditions comparable to those utilized to determine or analyze the characteristic or value of interest.

The term “sample” or “biological sample,” as used herein, refers to a biological sample obtained or derived from a source of interest, as described herein. In certain embodiments, a source of interest comprises an organism, such as a microbe, a plant, an animal or a human. In certain embodiments, a biological sample is or comprises biological tissue or fluid. In certain embodiments, a biological sample may be or comprise bone marrow; blood; blood cells; ascites; tissue or fine needle biopsy samples; cell-containing body fluids; free floating nucleic acids (e.g., cell free DNA); sputum; saliva; urine; cerebrospinal fluid, peritoneal fluid; pleural fluid; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In certain embodiments, a biological sample is or comprises cells obtained from an individual. In certain embodiments, obtained cells are or include cells from an individual from whom the sample is obtained. In certain embodiments, a sample is a “primary sample” obtained directly from a source of interest by any appropriate means. For example, in certain embodiments, a primary biological sample is obtained by methods selected from the group consisting of a swab, biopsy (e.g., fine needle aspiration or tissue biopsy), surgery, collection of body fluid (e.g., blood, lymph, feces etc.), etc. In certain embodiments, as will be clear from context, the term “sample” refers to a preparation that is obtained by processing (e.g., by removing one or more components of and/or by adding one or more agents to) a primary sample. For example, filtering using a semi-permeable membrane. Such a processed “sample” may comprise, for example nucleic acids or proteins extracted from a sample or obtained by subjecting a primary sample to techniques such as amplification or reverse transcription of mRNA, isolation and/or purification of certain components, etc.

As used herein, the term “specific” means exclusively present, absent, relatively enriched and/or relatively depleted compared to an appropriate reference. For example, a cell type-specific transcript (e.g., a rare cell-specific transcript) is or comprises a transcript (e.g., an RNA copy of a sequence, or portion thereof) that is exclusively present, absent, relatively enriched and/or relatively depleted in a particular cell type relative to an appropriate reference (e.g., a different cell type).

As used herein, the term “specifically hybridize” or “has selective affinity for” means a nucleic acid (e.g., a nucleic acid probe) hybridizes or associates more frequently, more rapidly, with greater duration, with greater affinity, or combinations thereof to a particular target molecule (e.g., a target nucleic acid of interest, a cell type-specific transcript) than with alternative molecules, including unrelated molecules (e.g., non-target nucleic acids). It is understood that, in certain embodiments, a nucleic acid (e.g., a nucleic acid probe) that specifically hybridizes with a first target molecule may or may not specifically hybridize with a second target molecule. As such, “specific hybridization” does not necessarily require (although it can include) exclusive hybridization, i.e., hybridization to a single target molecule. Thus, a nucleic acid (e.g., a nucleic acid probe) may, in certain embodiments, specifically hybridize with more than one target molecule.

As used herein, the term “subject” or “patient” refers to any organism upon which embodiments of the invention may be used or administered, e.g., for experimental, screening, diagnostic, prophylactic, and/or therapeutic purposes. Typical subjects include animals (e.g., mammals such as mice, rats, rabbits, non-human primates, and humans; insects; worms; etc.).

As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### Samples

In some embodiments, a sample (e.g., a biological sample) for use in accordance with the present disclosure is or comprises a sample obtained or derived from a source of interest. In some embodiments, a source of interest comprises an organism, such as a microbe, a plant, an animal, or a human. In some embodiments, a sample is or comprises a clinical sample obtained from a subject (e.g., a human, non-human primate, mouse, dog, cat, cow, horse, poultry, reptile, fish). In some embodiments, the sample is obtained from a human. In some embodiments, the human may be pregnant.

In some embodiments, a biological sample is or comprises biological tissue and/or fluid. In some embodiments, a biological sample may be or comprise bone marrow; blood or a fraction thereof (e.g., serum, plasma, buffy coat); blood cells; ascites; tissue or fine needle biopsy samples; cell-containing body fluids; sputum; saliva; urine; cerebrospinal fluid; peritoneal fluid; pleural fluid; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, the biological sample may be selected from whole blood, plasma, and serum.

In some embodiments, a biological sample may be or comprise whole blood, buffy coat, plasma, serum, peripheral blood mononucleated cells (PBMCs), band cells, neutrophils, monocytes, or T cells. In some embodiments, a biological sample may be or comprise maternal blood or a fraction thereof, such as buffy coat. In some embodiments, a biological sample may be or comprise placental cells (e.g., a sample of enriched placental cells).

### Rare Cells

In some embodiments, a rare cell is or comprises a cell type with low abundance relative to an appropriate reference (e.g., total cells in a sample, volume of a sample). In some embodiments, a rare cell is present in a sample at an abundance of about 1, 2, 3, 4, 5, 6, 7, 8, 9, 10, 15, 20, 25, 50, 75, 100, 150, 200, 250, 500, 750, 1,000, 1,250, 1,500, 1,750, or 2,000 cells/mL of sample. In some embodiments, a rare cell is present in a sample at an abundance of about 1-500 cells/mL of sample, 1-1,000 cells/mL of sample, 1-1,500 cells/mL of sample, 1-2,000 cells/mL of sample, 500-2,000 cells/mL of sample, 500-1,500 cells/mL of sample, or 1,000-2,000 cells/mL of sample. In some embodiments, a rare cell is present in a sample at an abundance of about 1e⁻⁸%, 1e⁻⁷%, 1e⁻⁶%, 1e⁻⁵%, 1e⁻⁴%, 0.001%, 0.005%, 0.01%, 0.05%, 0.1%, 0.5%, or 1% of the total number of cells in the sample. In some embodiments, a rare cell is present in a sample at an abundance of about 1e⁻⁸%-1%, 1e⁻⁷%-1%, 1e⁻⁶%-1%, 1e⁻⁵%-1%, 1e⁻⁴%-1%, 0.001%-1%, 0.005%-1%, 0.01%-1%, 0.05%-1%, 0.1%-1%, 0.5%-1%, 0.001%-0.5%, 0.001%-0.1%, 0.001%-0.05%, 0.001%-0.01%, 0.001%-0.005% or 1e⁻⁸%-1e⁻⁴% of the total number of cells in the sample.

In some embodiments, rare cells for use in accordance with the technologies of the present disclosure, include, for example and without limitation, basophils, cells of fetal trophoblast origin, circulating embryonic stem cells, circulating endothelial cells, circulating epithelial cells, circulating erythroblasts, circulating fetal cells (CFCs), circulating hematopoietic stem cells, circulating megakaryocytes, and circulating trophoblasts. In some embodiments, the rare cells may be fetal trophoblasts. In some embodiments, the rare cells may be CFCs.

### Nucleic Acid Probes

The present disclosure provides technologies that utilize nucleic acid probes that hybridize (e.g., specifically hybridize) to target nucleic acids of interest (e.g., cell type-specific transcripts described herein). In some embodiments, a nucleic acid probe comprises a nucleotide sequence complementary to a target nucleic acid of interest (e.g., a hybridization sequence). In some embodiments, a nucleic acid probe comprises a hybridization sequence about 70%, 75%, 80%, 85%, 90%, 91%, 92%, 93%, 94% 95%, 96%, 97%, 98%, 99% or 100% complementary to a target nucleic acid of interest (e.g., a cell type-specific transcript). In some embodiments, technologies of the present disclosure utilize one or more nucleic acid probes. In some embodiments, one or more nucleic acid probes specifically hybridize to the same target nucleic acid of interest. In some embodiments, one or more nucleic acid probes specifically hybridize to a plurality of different target nucleic acids of interest. In some embodiments, technologies of the present disclosure utilize a plurality of nucleic acid probes that specifically hybridize to a plurality of different target nucleic acids of interest (e.g., a pooled probe set). In some embodiments, a nucleic acid probe is a variant-specific nucleic acid probe (e.g., specifically hybridizes to a target nucleic acid of interest comprising a variant). In some embodiments, a nucleic acid probe further comprises a detectable marker.

In some embodiments, a nucleic acid probe comprises an In Situ Hybridization (ISH) probe set. In some embodiments, a nucleic acid probe comprises a Fluorescence In Situ Hybridization (FISH) probe set. In some embodiments, an ISH probe set or a FISH probe set is biotinylated. In some embodiments, a nucleic acid probe comprises multiply labeled tetravalent RNA imaging probes (MTRIPs), including, for example, single-molecule sensitive probes for imaging RNA in live cells (see, e.g., Santangelo et al., Nat. Methods, 2009).

In some embodiments, a nucleic acid probe comprises a hybridization chain reaction (HCR) probe set. In some embodiments, a HCR probe set comprises one or more initiator probes each comprising a hybridization sequence. In some such embodiments, a HCR probe set comprises at least two initiator probes each comprising hybridization sequence complementary to a target nucleic acid of interest at a position adjacent to that where another initiator probe hybridizes (e.g., a split-initiator). In some embodiments, a HCR probe set further comprises at least two HCR amplifiers. In some such embodiments, a HCR amplifier comprises a metastable hairpin comprising a sequence complementary to an initiator probe and/or another HCR amplifier and a detectable marker (e.g., a fluorophore). Without wishing to be bound by any one theory, HCR amplifiers'kinetically trapped, hairpin structure store energy to drive a conditional self-assembly cascade upon exposure to a cognate initiator probe (e.g., an initiator probe comprising a sequence complementary to a portion of an HCR amplifier). Upon hybridization of a first HCR amplifier to a cognate initiator probe, the first HCR amplifier's hairpin structure opens exposing an output domain which hybridizes to the input domain of the second HCR amplifier. Upon hybridization, the second HCR amplifier's hairpin structure opens exposing an output domain which is identical in sequence to the first initiator probe sequence, thus providing the basis for a chain reaction of alternating first and second HCR amplifier polymerization steps. Design and use of HCR probe sets are readily understood and known in the art (see, e.g., WO2021221789, Choi H. M. T. et al., Development (2018) 145, dev 165753). One of ordinary skill in the art, reading the present disclosure, would readily recognize and understand how to select, design, and/or use HCR probe sets in accordance with technologies of the present disclosure.

A plurality of methods are known in the art to design nucleic acid probes. One of ordinary skill in the art, reading the present disclosure, would readily recognize and understand how to select and use such methods in accordance with technologies of the present disclosure. See, for example, WO2007064758, WO2004025257, WO2017147702,WO2005051967, Wu et al., Nat Methods. 2015 Dec; 12(12): 1191-1196, Zhang et al., Nat Chem. 2012 Mar; 4(3): 208-214.).

### Cell Type-Specific Transcripts

Transcriptional signatures (e.g., gene expression patterns characteristic of a particular cell type, disease state, etc.) and cell type-specific transcripts can be used, for example, to diagnose disease status and/or prognosis in a given subject and thus, guide treatment decisions, in understanding diseases mechanisms, and/or to discriminate between cell types. In some embodiments, a cell type-specific transcript is or comprises a transcript (e.g., an RNA copy of a sequence, or portion thereof) that is exclusively present, absent, relatively enriched and/or relatively depleted in a particular cell type relative to an appropriate reference (e.g., a different cell type). In some embodiments, a transcriptional signature comprises a plurality of cell type-specific transcripts.

In some embodiments, a cell type-specific transcript is or comprises a transcript that is present and/or relatively enriched in a particular cell type relative to an appropriate reference (e.g., a different cell type). In some embodiments, a cell-type specific transcript is enriched relative to an appropriate reference by a factor of about 1.5, 2, 3, 4, 5, 6, 7, 8, 9, 10, 15, 20, 25, 30, 35, 40, 45, 50, 60, 70, 80, 90, 100, 250, 500, 750, 1000, 2000, 3000, 4000, 5000, 6000, 7000, 8000, 9000, or 10000. In some embodiments, a cell-type specific transcript is enriched relative to an appropriate reference by a factor of about 1.5-1,000, 2-1,000, 5-1,000, 10-1,000, 50-1,000, 100-1,000, 500-1,000, 1.5-750, 2-750, 5-750, 10-750, 50-750, 100-750, 1.5-500, 2-500, 10-500, 50-500, 100-500, 250-500, 500-10000, 1000-10000, or 5000-10000.

In some embodiments, a cell type-specific transcript is or comprises a transcript that is absent and/or relatively depleted in a particular cell type relative to an appropriate reference (e.g., a different cell type). In some such embodiments, a cell type-specific transcript is depleted relative to an appropriate reference by a factor of about 0.5, 0.4, 0.3, 0.2, 0.1, 0.05, 0.01, 0.005, 0.001, 0.0005, or 0.0001. In some such embodiments, a cell type-specific transcript is depleted relative to an appropriate reference by a factor of about 0.5-0.0001, 0.5-0.0005, 0.5-0.001, 0.5-0.005, 0.5-0.01, 0.5-0.05, 0.5-0.1, 0.1-0.0001, 0.1-0.0005, 0.1-0.001, 0.1-0.005, 0.1-0.01, 0.1-0.05, 0.05-0.0001, 0.01-0001 or 0.01-0.0005.

In some embodiments, a cell type-specific transcript is identical to that of a transcript of appropriate reference (e.g., a different cell type), except for the relative absence or presence of a variant.

In some embodiments, a plurality of nucleic acid probes (e.g., 2, 3, 4, 5, 6, 8, 10, 12, or more nucleic acid probes) comprise hybridization sequences complementary to a plurality of cell-type specific transcripts (e.g., 2, 3, 4, 5, 6, 8, 20, 12 or more cell type-specific transcripts, e.g., a transcriptional signature). In some such embodiments, the plurality of cell-type specific transcripts are exclusively present, absent, relatively enriched, and/or relatively depleted in a specific cell-type relative to an appropriate reference (e.g., a different cell type) by different factors. For example, one cell-type specific transcript may be enriched by a factor of two relative to an appropriate reference and a second cell-type specific transcript is depleted by a factor of 0.01 relative to an appropriate reference. In some such embodiments, a plurality of cell-type specific transcripts are exclusively present, absent, relatively enriched, and/or relatively depleted in a specific cell-type relative to an appropriate reference by similar factors. For example, one cell-type specific transcript may be enriched by a factor of two relative to an appropriate reference and a second cell-type specific transcript may also be enriched by a factor of two relative to an appropriate reference.

In some embodiments, presence, absence and/or level of a cell type-specific transcript and/or a transcriptional signature is characteristic of a particular state and/or event. In some such embodiments, presence, absence and/or level of a particular cell type-specific transcript and/or transcriptional signature may be characteristic of presence, absence and/or stage of a disease, disorder, and/or condition. Alternatively or additionally, in some embodiments, presence and/or level of a particular cell type-specific transcript correlates with activity (or activity level) of a particular signaling pathway, for example, that may be characteristic of a particular cell type, disease, disorder, and/or condition.

In some embodiments, a cell type-specific transcript is a transcript unique (e.g., present, absent) and/or relatively enriched or depleted in a rare cell (e.g., as described herein) relative to an appropriate reference. In some embodiments, one or more cell type-specific transcripts make up transcriptional signature unique and/or relatively enriched or relatively depleted in a particular cell type (e.g., rare cells described herein). In some embodiments, a cell type-specific transcript is or comprises, for example, a basophil-specific transcript, a circulating embryonic stem cell-specific transcript, a circulating endothelial cell-specific transcript, a circulating epithelial cell-specific transcript, a circulating erythroblast-specific transcript, fetal cell-specific transcript, including, for example, circulating fetal cells (see, e.g., Cao J et al., Science. 2020 November 13;370(6518)), a circulating hematopoietic stem cell—23-specific transcript, a circulating megakaryocyte-specific transcript, a circulating trophoblast-specific transcript, a maternal cell-specific transcript (e.g., of a pregnant woman), or a paternal cell-specific transcript.

In some embodiments, a cell-type specific transcript is a transcript unique (e.g., present, absent) and/or relatively enriched or depleted in a cell (e.g., a rare cell, a fetal cell) of a particular patient (e.g., a patient-specific transcript) relative to an appropriate reference.

In some embodiments, a cell-type specific transcript is or comprises, for example and without limitation, CSH1, CSH2, DDX3Y, EIF1AY, IGHG4, KDM5D, MIR4280HG, NLGN4Y, PAEP, PRKY, RPS4Y1, TBLIY, TMSB4Y, USP9Y, UTY, ZFY.

### Detectable Markers

In some embodiments, a nucleic acid probe, including, for example, a HCR amplifier, further comprises a detectable marker. In some embodiments, a detectable marker is detectable (e.g., fluorescent, luminescent) upon hybridization of the nucleic acid probe to the target nucleic acid of interest. In some embodiments, a nucleic acid probe comprises zero, one, or more (e.g., 2, 3, 4, 5, 6) detectable markers. In some embodiments, different nucleic acid probes comprise different detectable markers. In some embodiments, different nucleic acid probes comprise the same detectable markers. In some embodiments, one or more detectable markers of a given nucleic acid probe can be unique within a mixture of nucleic acid probes and/or detectable markers. In some embodiments, there are 1, 10, 1,000, 10,000, 100,000 or more unique detectable markers within a mixture (e.g., including any range defined between any two of the previous numbers).

In some embodiments, a detectable marker is a molecule that facilitates measurement of a signal (e.g., fluorescent signal). In some embodiments, a detectable marker is or comprises a fluorophore, a chromophore, a luminophore, a phosphor, a FRET pair, a member of a FRET pair, a quencher, a fluorophore/quencher pair, a magnetic molecule, or any other molecule that facilitates measurement of a signal and can be conjugated to a nucleic acid probe as described herein. Exemplary fluorophores include, without limitation, DyLight 405, Alexa Fluor 405, Pacific Blue, Alexa Fluor 488, fluorescein isothiocyanate (FITC), DyLight 550, Allophycocyanin (APC), Phycoerythrin (PE), peridinin chlorophyll protein (PerCP), Alexa Fluor 647, DyLight 650, Alexa Fluor 700, StarBright Violet 440, StarBright Violet 515, StarBright Violet 610, StarBright Violet 670, StarBright Violet 700, PE-Alexa Fluor 647, PE-Cy5, PerCP-Cy5.5, PE-Cy5.5, PE-Alexa Fluor 750, PE-Cy7, APC-Cy7, Green Fluorescent Protein (GFP), enhanced GFP (eGFP), Cyan Fluorescent Protein (CFP), Yellow Fluorescent Protein (YFP), Red Fluorescent Protein (RFP), and/or mCherry. In particular, fluorophores with an emission in the range of 488 and 647 may be utilized in the disclosed systems and methods.

In some embodiments, use of a plurality of nucleic acid probes (e.g., 2, 3, 4, 5, 10, 20, or more), each of which further comprising a different detectable marker, can be useful for multiplexing (e.g., detection and/or analyses of a plurality of nucleic acid targets of interest). In some embodiments, such technologies can be readily multiplexed to achieve simultaneous detection and/or analyses of a plurality of nucleic acid targets of interest (e.g., multiple cell type-specific transcripts, of the same or different cell types, or a transcript comprising a variant associated with a particular disease, disorder, and/or condition).

In some embodiments, one or more nucleic acid probes described herein comprises a hybridization sequence complementary to one or more cell type-specific transcripts (e.g., rare cell-specific transcripts) and a detectable marker. Without wishing to be bound by any one theory, contacting a sample with one or more nucleic acid probes comprising a hybridization sequence complementary to one or more cell type-specific transcripts (e.g., rare cell-specific transcripts) and a detectable marker, results in hybridization of the probe to the one or more cell type-specific transcripts and thereby detectably labels the cell type comprising the one or more cell type-specific transcripts.

### Exemplary Methods of Isolating Cells (e.g., Rare Cells)

In some embodiments, the present disclosure provides, among other things, methods of isolating cells (e.g., rare cells). In some embodiments, methods of isolating cells (e.g., rare cells) comprises detectably labeling the cells to be isolated (e.g., using nucleic acid probes comprising a detectable marker described herein) and separating the detectably labeled cells from one or more undesirable components. In some embodiments, detectably labeling the cells to be isolated comprises contacting a sample with one or more nucleic acid probes described herein comprising a nucleic acid sequence complementary to one or more target nucleic acids of interest (e.g., cell type-specific transcripts), wherein the nucleic acid probe comprises a detectable marker.

In some embodiments, cells (e.g., rare cells) are isolated using immunomagnetic cell separation (e.g., MACS), flow cytometry, and/or fluorescence-activated cell sorting (FACS).

In some embodiments, technologies of the present disclosure isolates cells (e.g., rare cells) from a sample to a certain degree of purity with respect to other components of a preparation (e.g., other cell types or sample components). In some embodiments, isolated cells and/or enriched populations of cells comprise cells (e.g., rare cells) that are at least about 1%, 2%, 3%, 4%, 5%, 10%, 15%, 20%, 25%, 30%, 40%, 50%, 55%, 60%, 65%, 70%, 75%, 80%, 85%, 90%, 95%, 99% or more pure, with respect to other components of a preparation.

FACS is a specialized type of flow cytometry that provides a method for sorting a heterogeneous mixture of cells and/or isolating a particular cell type (e.g., rare cells) from a heterogeneous mixture of cells based upon the specific light scattering and fluorescent characteristics of a particular cell (see, e.g., Agarwal et al., Biosc. Biotech. Res. Comm. Special Issue Vol 13 No 14 (2020) Pp-436-439). Such technologies can provide fast, objective, and/or quantitative recording of fluorescent signals from individual cells as well as physical separation (e.g., isolation) of cells of interest (e.g., rare cells). In some embodiments, use of FACS to separate (e.g., isolate) cells of interest (e.g., rare cells) is challenging due to, for example, endogenous auto-fluorescence of cells of interest and/or auto-fluorescence of cells in the heterogeneous mixture of cells and/or limited signal intensity of detectable markers (e.g., fluorophores).

Magnetic-Activated Cell Sorting (MACS) is an affinity-based technique also used for sorting a heterogeneous mixture of cells and/or isolating a particular cell type (e.g., rare cells) from a heterogeneous mixture of cells using magnetic particles functionalized to enable binding to a subset of cells in a mixture, thus facilitating separation. Typically, the magnetic particles are functionalized with an antibody specific for an antigen expressed on the surface of the cells of interest. In some embodiments, a probe (e.g., a nucleic acid probe) that specifically hybridizes to a cell-type specific transcript comprises a magnetic molecule or particle. The magnetic molecules or particles and the heterogeneous mixture of cells are incubated and subsequently placed in a magnetic field. Cells that do not express the antigen of interest or comprise the cell-type specific transcript are not retained in the magnetic field, whereas cells that do display the antigen of interest or comprise the cell-type specific transcript bind to the beads and are retained. Once the magnetic field is removed, the cells of interest can be eluted. See, e.g., Shen M J et al., ACS Appl Mater Interfaces. 2021 March 17; 13(10):11621-11630). In some embodiments, use of probes described herein (e.g., HCR probe set) amplifies the signal of a detectable marker to a level such that cells (e.g., rare cells) can be isolated using FACS or MACS. Without wishing to be bound by any one theory, amplification of detectable marker signal by use of HCR probes can increase sensitivity of FACS or MACS sorting (e.g., isolation of cells) to permit sorting (e.g., isolation) of rare cells. In some such embodiments, use of HCR probes amplifies the signal of a detectable marker by a factor of about 2, 3, 4, 5, 6, 7, 8, 9, 10, 15, 20, 25, 30, 35, 40, 45, 50, 100, 200, 300, 400, 500, 600, 700, 800, 900, 1,000, 1,100, 1,200, 1,300, 1,400, 1,500, 2,000, 5,000, 10,000, 2-1,000, 2-10,000, 10-100, 10-1,000, 10-10,000, or 100-10,000 relative to an appropriate reference (e.g., a detectable marker alone).

### Exemplary Methods of Characterizing Isolated Cells

The present disclosure, among other things, provides technologies for characterizing isolated cells (e.g., rare cells). In some embodiments, characterizing isolated cells comprises verifying the identity of the isolated cells (e.g., secondary validation of isolated cells based on cell type-specific identifiers, such as gene expression patterns and/or cell morphology) relative to an appropriate reference. A plurality of methods to verify the identity of isolated cells (e.g., isolated rare cells) are understood in the art. Such methods include, for example, verification based on cell morphology and genetic verification.

In some embodiments, verifying the identity of isolated cells (e.g., isolated rare cells) comprises genetic verification (e.g., secondary validation of a cell type based on genetic identifiers) relative an appropriate reference. A number of methods are understood in the art to verify the genetic identity of cells, including, for example, isolated cells. Such technologies include, for example and without limitation, karyotyping, DNA-based methods (e.g., polymerase chain reaction (PCR), short tandem repeat (STR) profiling, single nucleotide polymorphism (SNP) genotyping, DNA sequencing), and RNA-based methods (e.g., RNA sequencing).

In some embodiments, genetic verification comprises karyotyping isolated cells.

In some embodiments, genetic verification comprises use of Polymerase Chain Reaction (PCR) to amplify one or more cell type-specific sequences from the isolated cells. In some embodiments, PCR is quantitative PCR. In some embodiments, PCR amplicons of cell type-specific sequences are further characterized by DNA sequencing PCR amplicons and determining the presence, absence, and/or relative level (enrichment or depletion) of the cell type-specific sequences. In some embodiments, PCR amplicons of cell-type specific sequences are further characterized by gel electrophoresis and determining the presence, absence, and/or relative level (enrichment or depletion) of the cell type specific sequences.

In some embodiments, genetic verification comprises DNA sequencing isolated cells and detecting the presence, absence, and/or relative level (enrichment or depletion) of one or more cell type-specific sequences (e.g., a cell type-specific transcripts). In some embodiments, genetic verification comprises DNA sequencing isolated cells and detecting the presence or absence of one or more variants (e.g., SNP) associated with a particular cell type. In some embodiments, genetic verification comprises sequencing genomic DNA at high frequency variant (e.g., SNP) locations and detecting the presence or absence of one or more variants associated with a particular cell type. In some such embodiments, a variant is homozygous. In some such embodiments, a variant is heterozygous.

In some embodiments, genetic verification comprises STR profiling. STR profiling is an analytical DNA technique which PCR-amplifies variable microsatellite regions from a genomic DNA template, separates the PCR amplicons on a genetic analyzer, and uses software to analyze the resulting data and compare the data from one specimen to databases housing previously generated STR sets. The technology can depend on the simultaneous amplification of multiple stretches of polymorphic DNA within a single vessel. Repetitive DNA sequences with varying numbers of repeats, referred to as STR loci, are amplified using primers with differently colored fluorophores. These amplicons are distinguished by both size and color and the STR profile can be compared to a known and/or baseline STR profile for a particular cell type (see, e.g., Nims R W et al., In Vitro Cell Dev Biol Anim. 2010;46(10):811-819).

In some embodiments, genetic verification comprises SNP genotyping. A SNP is a genomic variant at a single base position in the DNA. Typically, SNPs are biallelic, although very rarely tri-or tetraallelic forms can be found. SNPs can serve as important genetic and/or physical markers for comparative study, including, for example genetic identification of cell types. In some embodiments, SNP genotyping comprises the generation of allele-specific products for SNPs of interest followed by their detection for genotype determination (e.g., genetic verification of isolated cells) (see, e.g., Kim et al., Annual Review of Biomedical Engineering, Vol. 9:289-320 (2007)).

A plurality of DNA sequencing methods are understood in the art. One of ordinary skill in the art, reading the present disclosure, would readily recognize and understand how to select and use such methods in accordance with technologies of the present disclosure. In some embodiments, DNA sequencing comprises, for example, Sanger Sequencing, long-read sequencing, and/or Next Generation Sequencing.

In some embodiments, genetic verification comprises RNA sequencing isolated cells and detecting the presence, absence, and/or relative level (enrichment or depletion) of one or more cell type-specific transcripts. A plurality of RNA sequencing methods are understood in the art. One of ordinary skill in the art, reading the present disclosure, would readily recognize and understand how to select and use such methods in accordance with technologies of the present disclosure. In some embodiments, RNA sequencing comprises, for example, mRNA sequencing, targeted RNA sequencing, ultra-low-input RNA sequencing, single-cell RNA-seq, RNA Exome Capture sequencing, total RNA sequencing, small RNA sequencing, and/or ribosome profiling.

In some embodiments, the genetic identity of the isolated cells is validated as the desired cell type. In some embodiments, the genetic identity of the isolated cells is not validated as the desired cell type (e.g., isolated cells are a different, undesired cell type and/or an undesired cell type is identified as present at an undesired level in the desired isolated cell type population (e.g., genetic verification identifies a population of isolated cells as impure)). In some such embodiments, isolated cells determined to be impure and/or comprising an undesired cell type are discarded. In some embodiments, after discarding an impure and/or undesired isolated cell type population, cell isolation is repeated from a sample which has not previously been subjected to technologies described herein.

In some embodiments, isolated cells (e.g., rare cells, and in particular, fetal cells) are characterized for allelic balance. Allelic balance (AB) is a measure of the proportion of sequencing reads covering a variant's (e.g., SNP's) location that support the presence of the variant. In isolated populations of cells (e.g., isolated fetal cells), the allelic balance at any particular locus (e.g., loci with high frequency of SNPs) can be homozygous for the reference (e.g., wild-type nucleotide sequence or nucleotide) (AB˜=0), homozygous for the alternative allele (e.g., comprise a variant relative to the reference, such as a SNP, on both alleles) (AB˜=1), or heterozygous (e.g., comprise a variant relative to the reference, such as a SNP, on one allele) (AB˜=0.5). As diploid fetal cells (e.g., isolated fetal cells) inherit one maternal allele and one paternal allele, isolated populations of cells can be determined as maternal or fetal cells, as being derived from more than one source (e.g., a mixed population of fetal and maternal cells), and particular variants (e.g., SNPs) can be determined as maternally or paternally inherited by characterizing allelic balance. For example, if a particular locus is heterozygous for a variant in a maternal cell and homozygous in a fetal cell, this can indicate a paternally inherited allele. Similarly, if a particular locus is homozygous in a maternal cell and heterozygous in a fetal cell, this can indicate a paternally inherited allele. Mixed populations of cells (e.g., a mixed population of maternal or fetal cells) will have an AB greater than 0, but less than about 0.5 or greater than about 0.5 and less than 1.

In some embodiments, a population of isolated cells is characterized as in allelic balance and is determined as a pure population of isolated cells and/or as cells from a single source. In some such embodiments, a population of isolated cells characterized as in allelic balance are utilized for detection of genetic variants (e.g., as described herein). In some embodiments, a population of isolated cells is characterized as in allelic imbalance and is determined as an impure population of isolated cells and/or from more than a single source. In some such embodiments, a population of isolated cells characterized as in allelic imbalance is not utilized for detection of genetic variants (e.g., as described herein). See, e.g., WO2012174378.

In such embodiments, isolated fetal cells determined to be an impure population of isolated fetal cells are discarded. In some embodiments, following discarding an impure population of isolated cells, cell isolation (e.g., rare cell isolation) is repeated from a sample which has not previously been subjected to technologies described herein.

### Exemplary Methods of Detecting Genetic Variants in Isolated Cells

The present disclosure, among other things, provides technologies for detecting the presence or absence of genetic variants in cells (e.g., isolated cells, isolated rare cells). In some embodiments, technologies of the present disclosure provide methods of effective and highly sensitive cell sorting (e. g, isolation, and in particular, isolation of rare cells) and subsequent diagnostic or detection assays, including, for example, detecting the presence or absence of a genetic variant in the isolated cells.

In some embodiments, methods of detecting the presence or absence of a genetic variant in a cell (e.g., isolated cells, isolated rare cells) comprises: (a) contacting a sample with one or more nucleic acid probes described herein; (b) separating the cells (e.g., rare cells) from one or more undesired sample components, thereby enriching the population of cells as described herein; (c) sequencing the enriched population of cells; and (d) detecting the presence or absence of the genetic variant based on the sequencing reads. In some embodiments, the enriched population of cells comprises a plurality of cells. In some embodiments, the enriched population of cells comprises a single cell. In some embodiments, sequencing the enriched population of cells comprises sequencing a plurality of cells. In some embodiments, sequencing the enriched population of cells comprises sequencing a single cell (e.g., single-cell sequencing).

In some embodiments, sequencing comprises Sanger sequencing. Sanger sequencing is a method of DNA sequencing that involves electrophoresis and is based on the random incorporation of chain-terminating dideoxynucleotides by DNA polymerase during in vitro DNA replication (see, e.g., Heather J M et al., Genomics. 2016;107(1):1-8).

In some embodiments, sequencing comprises Next Generation Sequencing (NGS). NGS can sequence from a small number of genes (e.g., targeted sequencing) to an entire genome. In some embodiments, NGS comprises whole-genome sequencing (WGS) which determines the sequences of DNA bases across an entire genome. In some embodiments, NGS comprises whole-exome sequencing (WES) which determines the sequence of DNA bases across an entire exome. In some embodiments, NGS comprises transcriptome sequencing (e.g., RNA sequencing, whole transcriptome sequencing) which provides sequencing information about coding and multiple noncoding forms of RNA. In some such embodiments, transcriptome (RNA) sequencing can assess variations and gene expression levels, including across the entire transcriptome. In some embodiments, Sanger sequencing is utilized to confirm a sequence determined by NGS. In some such embodiments, Sanger sequence is utilized to confirm the presence or absence of a variant detected by NGS.

In some embodiments, sequencing comprises long-read sequencing. Long-read sequencing technologies can generate long continuous sequences (e.g., ranging from about 1 kilobase to greater than 10 kilobases, ranging from about 10 kilobases to greater than 1 megabase in length) directly from native DNA. Such technologies can also readily traverse the most repetitive regions of the genomes (see, e.g., Logsdon G A et al., Nat Rev Genet. 2020 Oct; 21(10):597-614).

In some embodiments, targeted sequencing comprises sequencing one or more loci of interest. In some embodiments, sequencing is targeted sequencing of a plurality of loci. A loci of interest may be, for example, one or more genomic loci (e.g., a gene panel) associated with a particular disease, disorder, and/or condition. In some embodiments, targeted sequencing of one or more loci of interest comprises use of Sanger sequencing. In some embodiments, targeted sequencing of one or more loci of interest comprises use of NGS.

In some embodiments, after sequencing, sequencing data is subjected to quality assessment. In some such embodiments, quality assessment comprises removing contaminants, such as, for example, adapter sequences and/or poor quality sequencing reads. A plurality of bioinformatic methods and/or tools are known in the art to conduct quality assessment on sequencing data. One of ordinary skill, reading the present disclosure, would readily recognize and understand how to select and use such methods in accordance with technologies of the present disclosure. Exemplary quality assessment methods and/or tools include, without limitation, FastQC, Trimmomatic, and fastp (see, e.g., Andrews, S. “FastQC: a quality control tool for high throughput sequence data.” (2010); Bolger, A. M. et al., “Bioinformatics 30.15 (2014): 2114-2120; Chen, Shifu, et al., Bioinformatics 34.17 (2018): i884-i890).

In some embodiments, detecting the presence or absence of a genetic variant based on the sequencing reads comprises aligning the sequencing data to that of an appropriate reference (e.g., a reference genome, a reference gene panel, a reference sequence). A plurality of bioinformatics methods and/or tools are known in the art to align sequencing data to one or more appropriate references. One of ordinary skill, reading the present disclosure, would readily recognize and understand how to select and use such methods and/or tools in accordance with technologies of the present disclosure. Exemplary alignment tools include, without limitation, BWA (Li, H. et al., Bioinformatics 25.14 (2009): 1754-1760); Bowtie2 (Langmead, B. et al., Nature methods 9.4 (2012): 357-359), CUSHAW3 (Liu, Y. et al., PloS one 9.1 (2014): e86869), MOSAIK (Lee, W. et al., PloS one 9.3 (2014): e90581).

In some embodiments, following alignment of the sequence reads to that of an appropriate reference, variant calling is conducted. In some embodiments, variant calling involves comparing aligned reads to an appropriate reference and identifying the presence or absence of variants (e.g., SNPs), insertions, and/or deletions. In some embodiments, bioinformatics methods and/or tools are selected that can accurately call variants in heterogeneous samples. A plurality of bioinformatics methods and/or tools are known in the art to conduct variant calling, including those that can accurately call variants in heterogeneous samples. One of ordinary skill, reading the present disclosure, would readily recognize and understand how to select and use such methods and/or tools in accordance with technologies of the present disclosure. Exemplary variant calling methods and/or tools include, without limitation, Genome Analysis Tool Kit HaplotypeCaller (GATK-HC) (McKenna, A., et al. Genome research 20.9 (2010): 1297-1303); GATK mutect2; Samtools mpileup (Li, H., et al. Bioinformatics 25.16 (2009): 2078-2079); Freebayes (Garrison, E, et al., arXiv preprint arXiv: 1207.3907 (2012)); SNPSVM (O'Fallon, B. et al., Bioinformatics 29.11 (2013): 1361-1366); DeepVariant (Poplin, R., et al., Nature biotechnology 36.10 (2018): 983-987); varScan (Koboldt, D. et al., Current protocols in bioinformatics 44.1 (2013): 15-4); Torrent Variant Caller (TVC) (Life Technologies, Rockville, MD); Mutect2 (Benjamin D. et al., bioRxiv; 2019).

In some embodiments, post-alignment quality control is conducted. A plurality of bioinformatics methods and/or tools are known in the art to conduct post-alignment quality control. One of ordinary skill, reading the present disclosure, would readily recognize and understand how to select and use such methods and/or tools in accordance with technologies of the present disclosure. Exemplary post-alignment quality control methods/tools include, for example and without limitation, Picard HSMetrics.

Any alignment method and/or tool can be used with any variant calling method and/or tool. In some embodiments, use of a particular alignment method and/or tool in combination with a particular variant calling method and/or tool results in relatively improved concordance and/or performance (e.g., sensitivity). In some embodiments, alignment and variant calling methods and/or tools are combined to generate an automated workflow (e.g., pipeline). In some embodiments, a pipeline further comprises up and down stream methods and/or tools (e.g., quality assessment) to form a complete end to end solution. In some embodiments, a pipeline comprises a plurality of variant calling methods and/or tools. In some such embodiments, use of a plurality of variant calling methods and/or tools can increase sensitivity relative to an appropriate reference (e.g., sensitivity of a pipeline using a single variant calling method and/or tool).

The present disclosure, among other things, provides technologies for detecting the presence or absence of genetic variants in cells (e.g., isolated cells, isolated rare cells) without post-cell isolation processing (e.g., sequencing). In some such embodiments, nucleic acid probes comprising a detectable marker (e.g., as described herein) specifically hybridize to a cell type-specific transcript comprising a genetic variant (e.g., a variant-specific nucleic acid probe). In some such embodiments, specific hybridization of a variant-specific nucleic acid probe indicates the presence of the genetic variant. In some such embodiments, the absence of specific hybridization of a variant-specific nucleic acid probes indicates the absence of the genetic variant. In some such embodiments, the presence or absence of specific hybridization of a variant-specific nucleic acid probe is determined and/or measured by the detectable marker. In some such embodiments, the detectable marker is measured using FACS. In some embodiments, use of one or more variant-specific nucleic acid probes comprising one or more detectable markers can facilitate multiplexing (e.g., simultaneous determination of the presence or absence of one or more genetic variants).

### Uses

Those skilled in the art, reading the present disclosure, will immediately appreciate that provided technologies are useful in a wide range of contexts and can be applied in a variety of formats.

In some embodiments, technologies of the present disclosure can be used to isolate cells (e.g., rare cells). Isolation of cells (e.g., rare cells) is useful in many research and clinical applications, including, for example, molecular analysis of specific cell populations, biophysical analysis of specific cell populations, and some diagnostic and detection assays that require a step of isolating cells from a sample (e.g., whole blood) to avoid interference of other components in the sample.

In some embodiments, technologies of the present disclosure can be used for detection of any RNA, DNA (e.g., double stranded DNA, single stranded DNA), or variants (e.g., SNPs) from isolated cells (e.g., rare cells). This enables detection of a variety of cell types, microorganisms, and/or nucleic acids indicative of infection or associated with human health, animal health, and plant health.

In some embodiments, technologies described herein are useful for detecting the presence or absence of a genetic variant in a cell (e.g., rare cells). In some such embodiments, detecting the presence or absence of a genetic variant in a cell further comprises diagnosing a subject with a disease and/or disorder based on the presence or absence of the genetic variant. In some embodiments, detecting the presence or absence of a genetic variant in a cell can provide information useful in determining whether a subject has a disease, condition, and/or disorder (e.g., diagnostic information) or the likelihood that a subject will develop a disease, disorder, and/or condition (e.g., prognostic information).

In some embodiments, technologies of the present disclosure are useful in classifying a disease and/or condition into a phenotypic category or any category having significance with regard to prognosis of a disease, disorder, and/or condition and/or likely response to a treatment of the disease, disorder, and/or condition. In some embodiments, technologies of the present disclosure are useful in selecting a treatment for a disease, disorder, and/or condition. In some such embodiments, selection of a treatment comprises choice of a particular therapeutic agent, treatment modality (e.g., surgery), whether to withhold or delivery therapy, and/or dosing regimen (e.g., frequency or level of one or more doses of a particular therapeutic agent or combination of therapeutic agents).

In some embodiments, technologies described herein are useful for multiplexed detection and/or analysis of nucleic acid targets of interest. In some embodiments, such technologies can be readily multiplexed to achieve simultaneous detection and/or analyses of a plurality of nucleic acid targets of interest (e.g., multiple cell type-specific transcripts, of the same or different cell types, or a transcript comprising a variant associated with a particular disease, disorder, and/or condition). In some embodiments, use of multiplexing in accordance with technologies described herein can facilitate detection and/or analyses of transcriptional signatures. In some embodiments, detection and/or analyses of cell type-specific transcripts, variants, and/or transcriptional signatures can facilitate diagnosis and/or prognosis of a disease, disorder, and/or condition. In some such embodiments, detection and/or analyses of cell type-specific transcripts, variants, and/or transcriptional signatures can inform prevention and/or treatment strategy for a subject in need thereof.

In some embodiments, technologies of the present disclosure are useful in non-invasive prenatal screening. In some such embodiments, non-invasive prenatal screening is conducted at the time of isolating a cell of interest (e.g., circulating fetal cells). In some embodiments, non-invasive prenatal screening further comprises diagnosing a fetus with a disease and/or disorder or determining that a fetus is likely (e.g., at an increased or high risk) of having a disease and/or disorder based on the presence or absence of a genetic variant in the circulating fetal cells. In some embodiments, a genetic variant in the circulating fetal cells is determined to be maternally or paternally inherited (e.g., by utilizing maternal haplotyping, see, e.g., US20170321270).

### EXAMPLES

### Example 1: General Experimental Methods

Nucleated cell isolation and fixation: Circulating fetal cells are found in the buffy coat layer after centrifugation separates Red Blood Cells (RBCs), nucleated cells, and plasma. Whole blood was collected in Streck BCT tubes. Blood tubes were spun for 10 minutes at 1600 g, plasma was removed and discarded (or stored for other uses), and the buffy coat was carefully aspirated, avoiding the RBC layer beneath, and saved in 1.5 ml Eppendorf tubes. The cells were then pelleted (˜200 g) for five minutes and washed 3 times in 1× Phosphate Buffered Saline with Tween (PBST). Washed cells were then fixed in 4% formaldehyde+PBST solution for 1 hour, washed 3 time with 1× PBST, and stored at −20° C. in 70% Ethanol until ready to use.

Probe Binding and Amplification: RNA probes from Molecular Instruments were ordered to target placental/fetal enriched transcripts. These probe sets bind to transcripts of interest inside the nucleus of the permeabilized cell. Fixed buffy coat nucleated cells from the above section were washed 3 times with 1×PBST (˜200 g centrifugation steps), then resuspended in PBST and counted on a flow cytometer. 1×10⁶ cells were used for each probe/condition being tested, pelleted and supernatant discarded. The remainder of the labeling protocol used Molecular Instruments (MI) reagents for HCR following the “MI protocol for RNA FISH mammalian cells in suspension” protocol. In brief, the cell pellet was resuspended in probe hybridization buffer and pre-hybridized for 30 minutes at 37° C. HCR initiator probe sets were added to the sample of fixed cells and incubated at 37° C. overnight. After approximately 16 hours, the sample of fixed cells was washed, incubated in amplification buffer and pre-amplify for 30 minutes at room temperature, and at approximately 18 hours, HCR amplifiers were added to the sample. After addition of the HCR amplifiers, the samples were incubated overnight (>12 hours) in the dark at room temperature, then washed and filtered prior to analysis by FACS. Modifications to the MI protocol for RNA FISH protocol include: pooling HCR initiator probes, using 16 nM-64 nM initiator probe concentrations, and only using nucleic acid probe set comprising Alexa-647 detectable marker. At the end of the HCR protocol cells were resuspended in 1× PBS and analyzed and sorted on the same day. Negative controls included: autofluorescence (AF, no probe, no amplifier), non-specific detection (NSD, GFP(−) probe+Alexa647 amplifier), non-specific amplification (NSA, no probe+amplifier), (autofluorescence controls shown).

Fluorescence Activated Cell Sorting (FACS): Immediately prior to FACS, cell nucleii were labeled with DAPI in the flow sample tube, and cells were filtered. HCR labeled cells and unlabeled controls were run on the BD FACSAria. Gating parameters were based on the Alexa 647 signal background of unlabeled control cells (that had been put through the HCR protocol without probes) and DAPI positive cells. Typically, in exemplary circulating fetal cells, positive labeled cells showed an order of magnitude increase in Alexa 647 signal over control cells. These cells were collected into 300 μl PBS in 1.5ml Eppendorf tubes and kept at 4° C. for further processing. Negative cells with low Alexa 647 signal were also collected for controls in analysis.

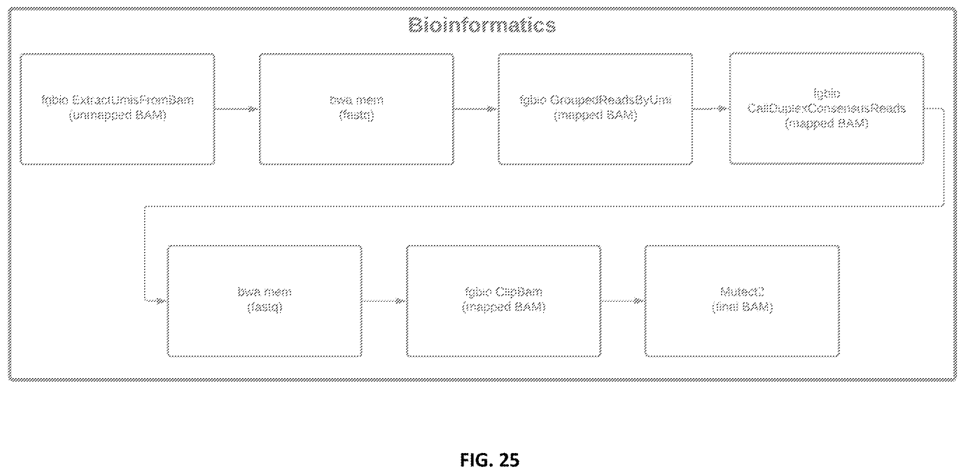

Cell type verification and downstream applications: Genotyping (cell type verification): Multiplex PCR primers were designed for 60 dbSNP sites for parent-of-origin determination, comparing fetal and maternal dbSNP sites. Labeled cells collected and sorted during FACS were pelleted at 300g for 15 minutes. Multiplexed amplification was performed on positive and negative control cells. Cells were then library prepped for NGS, and sequenced on Illumina iSeq. Data were analyzed as outlined in FIG. 25. Downstream applications: Positively labeled cells can then be used for downstream NGS methods. Since the yield of FACS positive cells was low (˜1-10 fetal cells per 1 million maternal cells), whole genome amplification was then performed. Samples were then library prepped for Illumina NGS and either whole genome sequenced, or processed for targeted sequencing.

### Example 2: Exemplary Isolation of Labeled Cells Using Hcr Probe Set

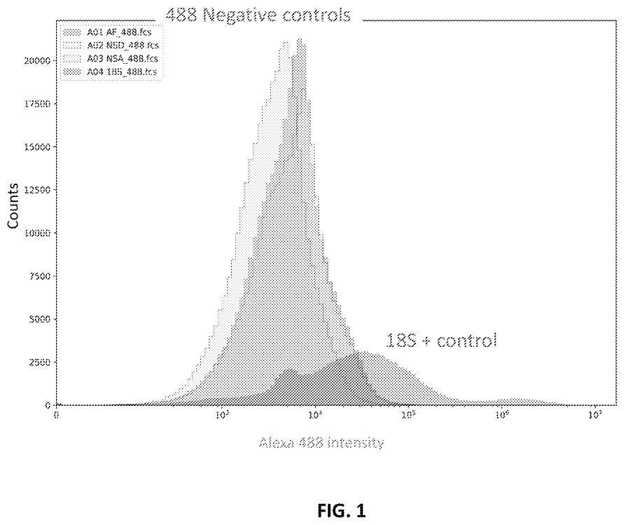

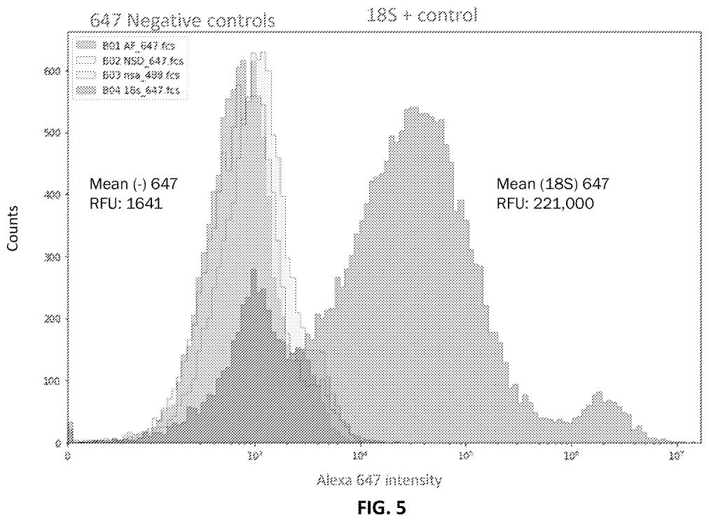

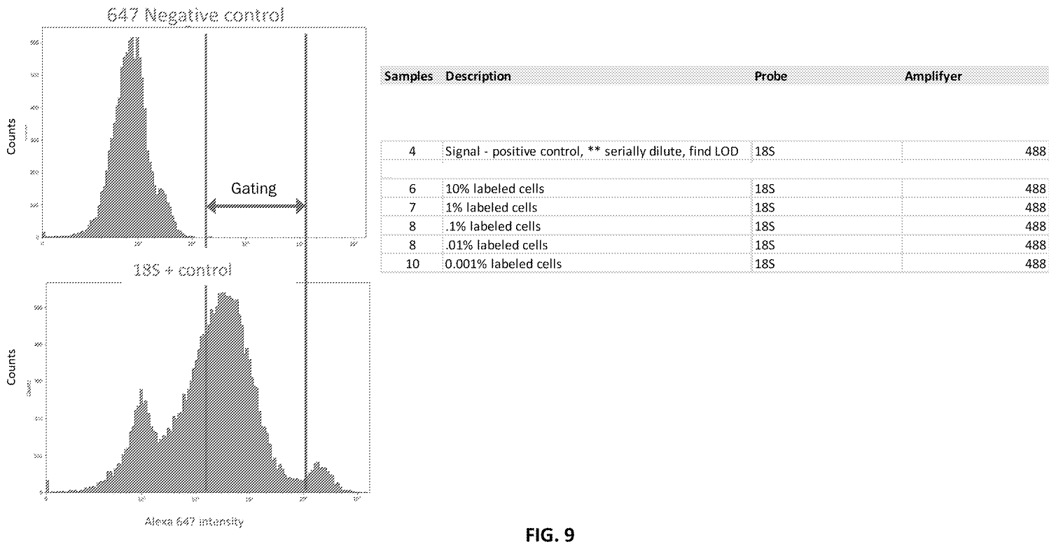

The present example demonstrates labelling with HCR probes and subsequent detection/isolation of labeled, low abundance cells (1-100 labeled cells in about 1 million unlabeled cells) using FACS. A HCR initiator probe set that specifically hybridizes to 18S ribosomal RNA was utilized to (1) assess the dynamic range of labelling with HCR probe sets followed by FACS, and (2) to determine the limit of detection using the 18S ribosome HCR probe set. Negative controls included samples that went through the entirety of the HCR protocol (see Probe Binding and Amplification above), but in the absence of HCR initiator and amplifier probes (Autofluorescence (AF) control), in the absence of 18S ribosomal RNA HCR initiator probes and the presence of an initiator probe that specifically hybridizes an RNA molecule absence in the sample and amplifier probes (Non-Specific Detection (NSD) control), or in the absence of initiator probes and the presence of amplifier probes (Non-Specific Amplification (NSA) control). Briefly, buffy coat cells were diluted to a level of about 1-10 labeled cells in about 1 million unlabeled cells. Diluted cells were labeled with 4 nM 18S ribosomal RNA HCR initiator probe set and the 18S ribosomal RNA HCR initiator probe set was amplified separately using 60 nM of HCR amplifier probes comprising either of Alexa Fluor 488 or Alexa Fluor 647 dyes. Labeled samples and controls were then detected/isolated using FACS. An increase in fluorescence intensity of cells labeled with the 18S ribosome HCR probe set was observed relative to negative controls (FIG. 1) and an increase in fluorescence intensity was observed relative to control (Alexa 647 amplifier only) (FIG. 5, FIG. 9).

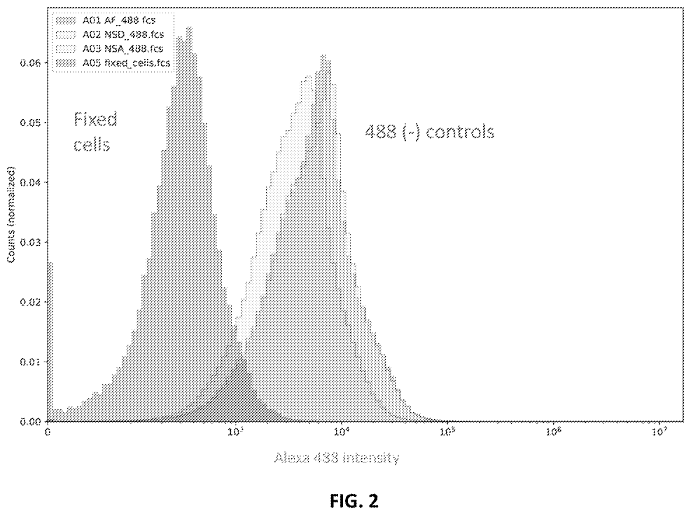

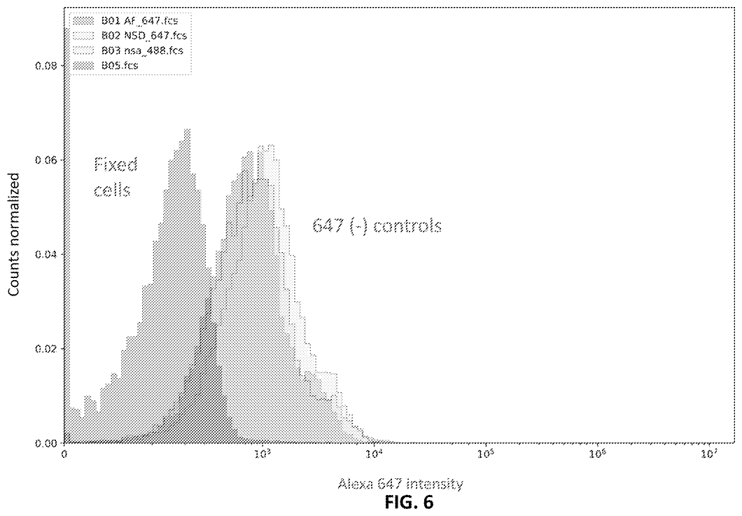

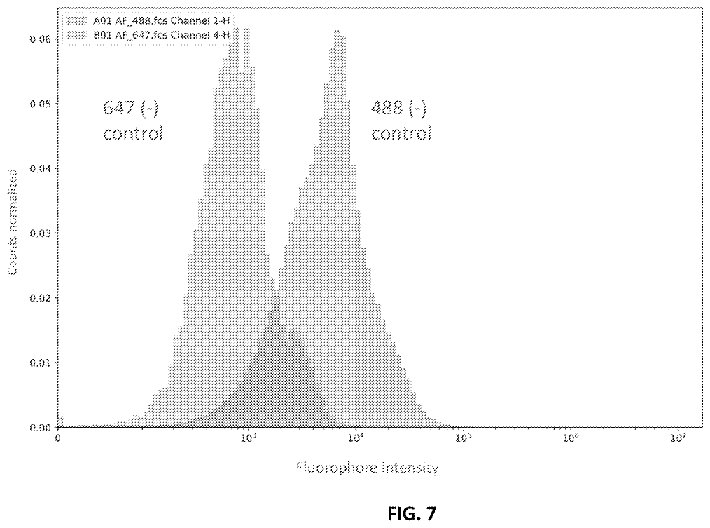

The impact of background fluorescence using the 18S ribosomal RNA HCR initiator probe set and either of an amplifier probe comprising an Alexa Fluor 488 detectable marker or an amplifier probe comprising an Alexa Fluor 647 detectable marker followed by FACS. Background fluorescence is likely from buffers when using an amplifier probe comprising an Alexa Fluor 488 detectable marker (FIG. 2). Background fluorescence is comparable to that of fixed cells when using an amplifier probe comprising an Alexa Fluor 647 detectable marker (FIG. 6). Use of a HCR amplifier probe comprising an Alexa Fluor 647 detectable marker has lower background than that of a HCR amplifier probe comprising an Alexa Fluor 488 detectable marker (FIG. 7).

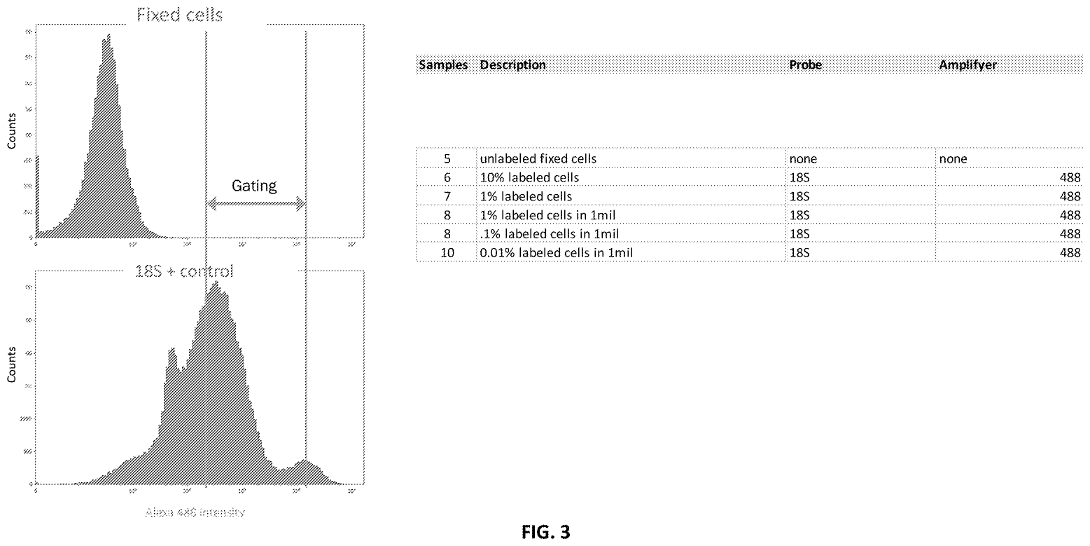

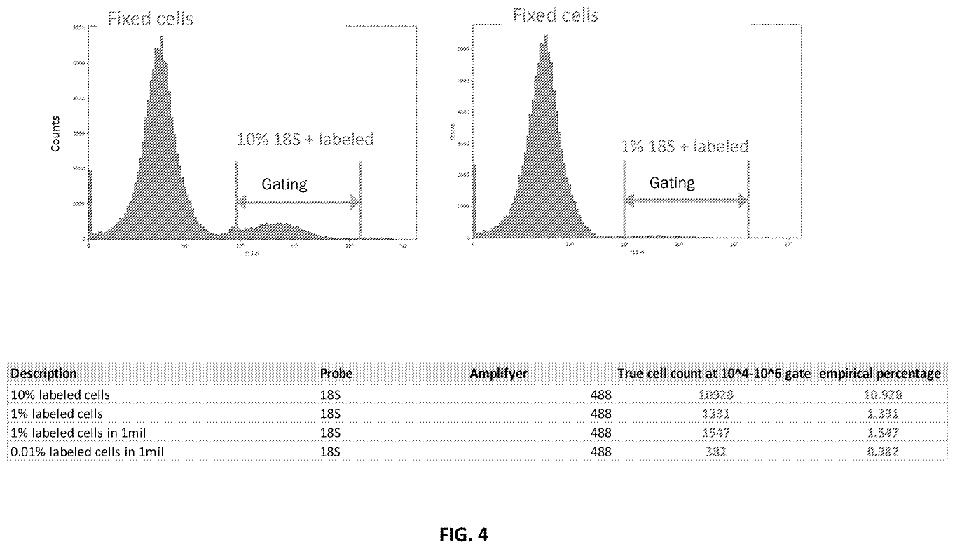

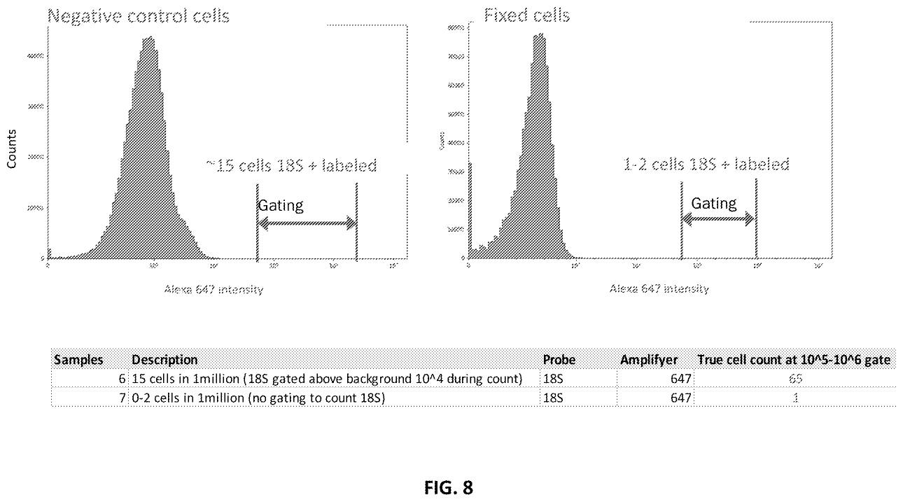

The level of detection (LOD) was also measured the 18S ribosomal RNA HCR initiator probe set and either of an amplifier probe comprising an Alexa Fluor 488 detectable marker or an amplifier probe comprising an Alexa Fluor 647 detectable marker followed by FACS. Use of either amplifier probe resulted in detection of diluted cells about 1 labeled cell in about 1 million unlabeled cells (FIG. 3, FIG. 4, FIG. 8).

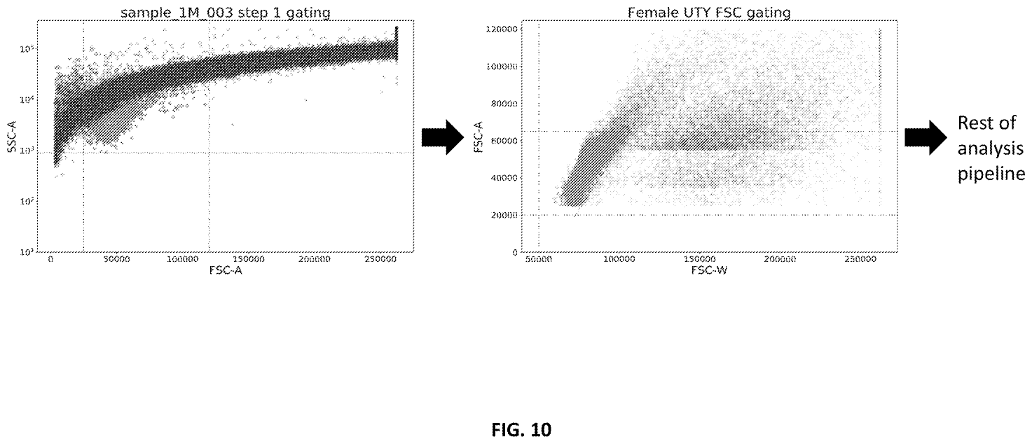

Without wishing to be bound by any one theory, it is understood that the HCR protocol can require harsh conditions, such as fixation, and as such, the majority of cells detected by flow cytometry (e.g., FACS) are dead cells and cellular debris. Accordingly, the importance of gating to remove dead cells and cellular debris was evaluated. Cells were labeled using a UTY-specific HCR probe set. HCR labelled cells were assessed by FACS using two gating steps. The first gating step removed cellular debris and 2-4.5% of the cells remained following the first gating step. The second gating step then removed doublet and higher-order cells. Of the 2-4.5% of the cells remaining from the first gating step, 15-82% of the cells remained following the second gating step (FIG. 10). Thus, gating to remove dead cells and cellular debris can be important for cleaning flow cytometry data. The cell population, with dead cells and debris removed, can then be characterized and the presence or absence of genetic variants can be assessed.

### Example 3: Exemplary Isolation of Circulating Fetal Cells (CFCs) Using Y-chromosome Specific-Nucleic Acid Probes

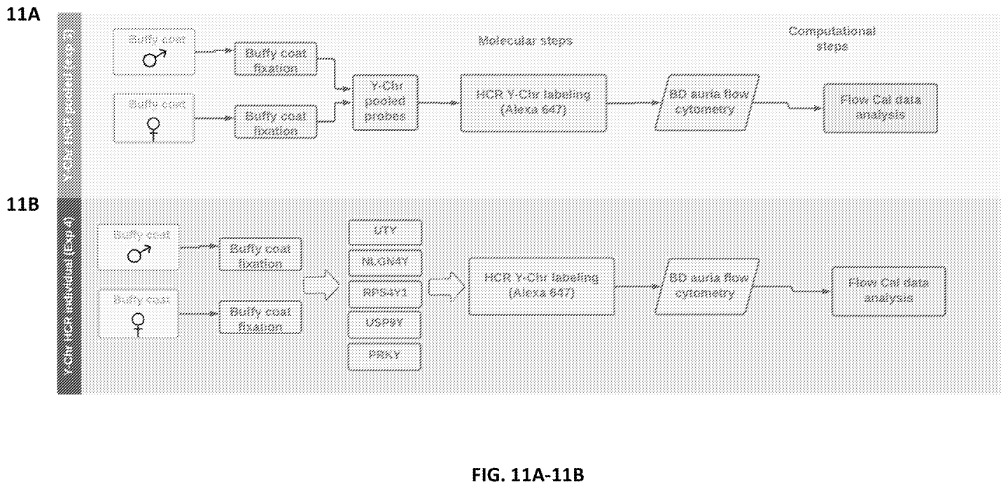

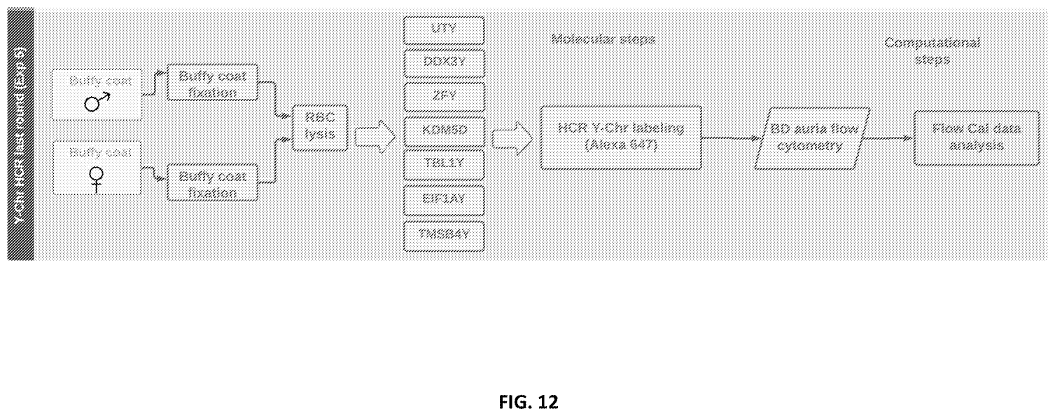

The present example demonstrates exemplary detection/isolation of CFCs using Y-chromosome specific-nucleic acid HCR probes followed by FACS. Y-chromosome specific-nucleic acid probes can include nucleic acid probes specific for, for example, UTY, DDX3Y, ZFY, KDM5D, TBLIY, EIF1AY, TMSB4Y, NLGN4Y, RPS4Y1, USP9Y, and/or PRKY. Pooled Y-chromosome specific nucleic acid probes can include two of more nucleic acid probes specific for, for example, UTY, DDX3Y, ZFY, KDM5D, TBLIY, EIF1AY, TMSB4Y, NLGN4Y, RPS4Y1, USP9Y, and/or PRKY. Exemplary workflows for isolation of CFCs using Y-Chromosome specific HCR probes are shown in FIGS. 11A-11B and FIG. 12.

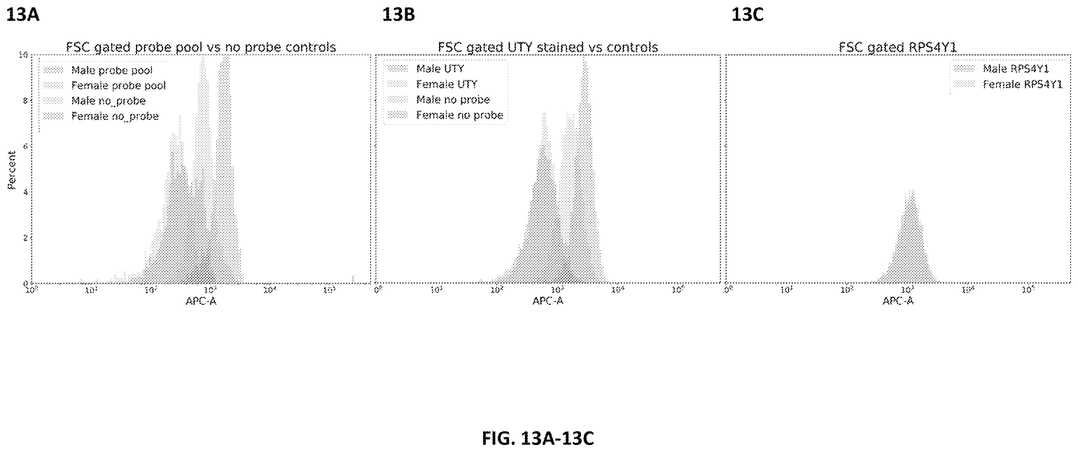

To evaluate Y-chromosome specific-nucleic acid probes, CFCs from male and female-derived buffy coat were labeled by HCR labelling with either pooled Y-chromosome specific-nucleic acid probes, UTY-specific nucleic acid probes, or RPS4Y1-specific nucleic acid probes. Samples that went through the entirety of the HCR protocol, but in the absence of HCR initiator probes (amplifier probes only), were utilized as a negative control. UTY-specific nucleic acid probes appeared to only slightly discriminate between male and female-derived cells (FIG. 13B), while RPS4Y1-specific nucleic acid probes did not discriminate between male and female-derived cells (FIG. 13C). Without wishing to be bound by any one theory, it is understood low discrimination between male and female-derived cells may be due to low abundance of chromosome Y transcripts in placental cells and/or labeling closely related and/or homologous X-chromosome gene transcripts. Use of pooled Y-chromosome specific-nucleic acid probes increased discrimination between male and female-derived cells (FIG. 13A). Chromosome Y labeling with HCR probes, with or without FACS, may be useful in secondary validation of isolated cells.

### Example 4: Exemplary Isolation of Putative CFCs Using Fetal/Placental Cell-Specific Nucleic Acid Probes

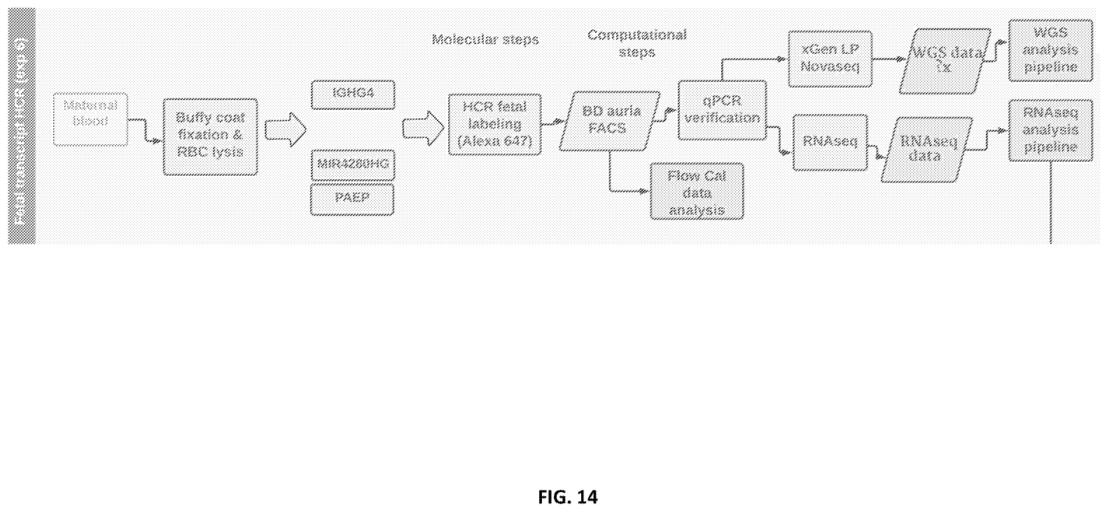

The present example demonstrates exemplary detection/isolation of putative CFCs using fetal/placental cell-specific nucleic acid probes. An exemplary workflow for isolation of such cells and subsequent detection of the presence or absence of a genetic variant is shown in FIG. 14.

HCR probe sets targeting single fetal/placental cell-specific transcripts (IGHG4, MIR4280HG, PAEP) were utilized in addition to a pooled HCR probe set which comprised HCR probe sets targeting each of IGHG4, MIR4280HG, and PAEP. HCR probes further comprised a far-red detectable marker. Samples that went through the entirety of the HCR protocol, but in the absence of HCR probes, were utilized as a negative control.

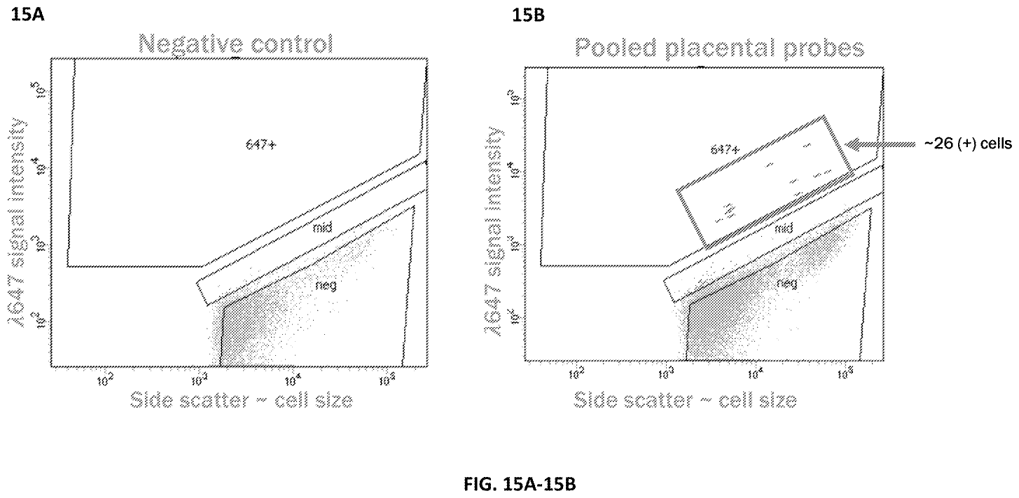

The pooled HCR probe set was added to the maternal blood sample to a final concentration of 64 nM. No putative CFCs were detected/isolated in the negative control group (FIG. 15A). The pooled HCR probe set detected/isolated 26 putative CFCs from the isolated buffy coat of maternal blood (FIG. 15B).

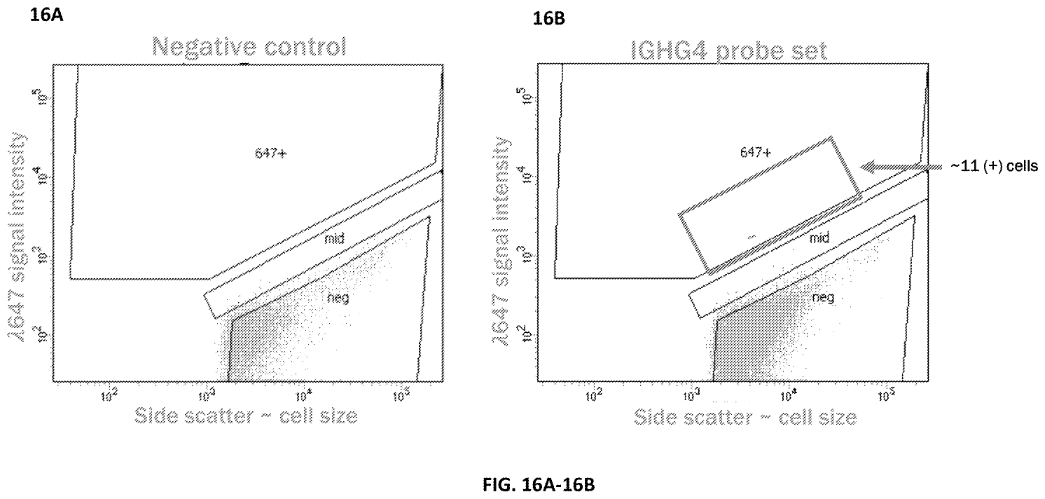

An HCR probe set that specifically hybridized to fetal/placental-specific transcript, IGHG4, was also evaluated at a final concentration of 16 nM. No putative CFCs were detected/isolated in the negative control group (FIG. 16A). The IGHG4 probe set detected/isolated 11 putative CFCs from the isolated buffy coat of maternal blood (FIG. 16B).

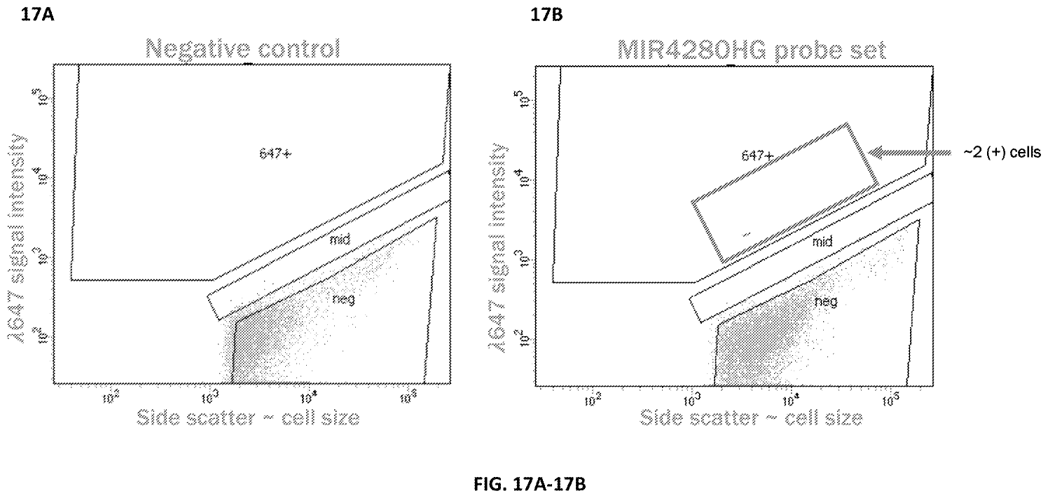

An HCR probe set that specifically hybridized to fetal/placental-specific transcript, MIR4280HG, was also evaluated at a final concentration of 16 nM. No putative CFCs were detected/isolated in the negative control group (FIG. 17A). The MIR4280HG probe set detected/isolated 2 putative CFCs from the isolated buffy coat of maternal blood (FIG. 17B).

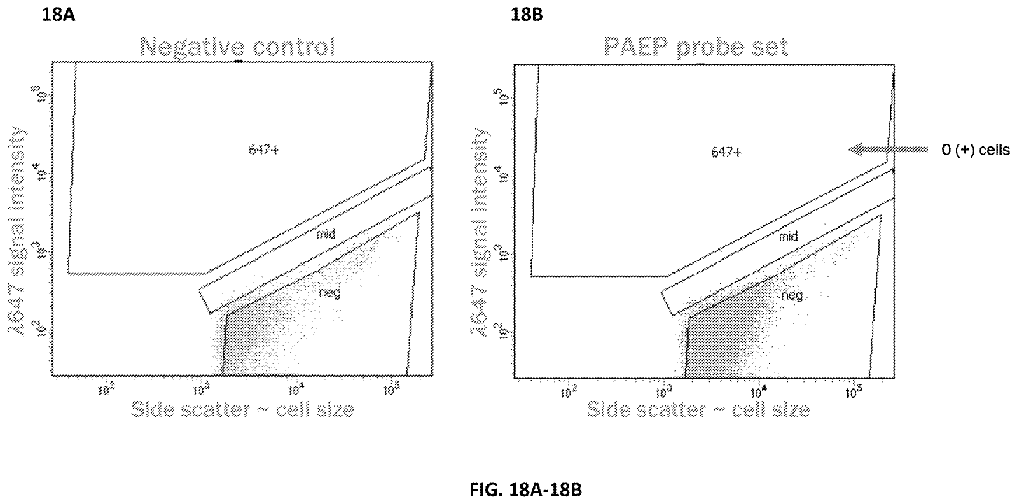

An HCR probe set that specifically hybridized to maternal-specific transcript, PAEP, was also evaluated as a secondary negative control at a final concentration of 16 nM. No putative CFCs were detected/isolated in the negative control group (FIG. 18A). The PAEP probe set also detected/isolated no putative CFCs from the isolated buffy coat of maternal blood (FIG. 18B).

Thus, the present example suggests detection/isolation of putative CFCs from maternal blood utilizing a pooled HCR probe set can increase sensitivity of detection/isolation relative to use of a single HCR probe set directed to a single fetal/placental cell-specific transcript.

### Example 5: Exemplary Isolation of Putative CFCs Using Fetal/Placental Cell-Specific Nucleic Acid Probes (CSH-1/2)

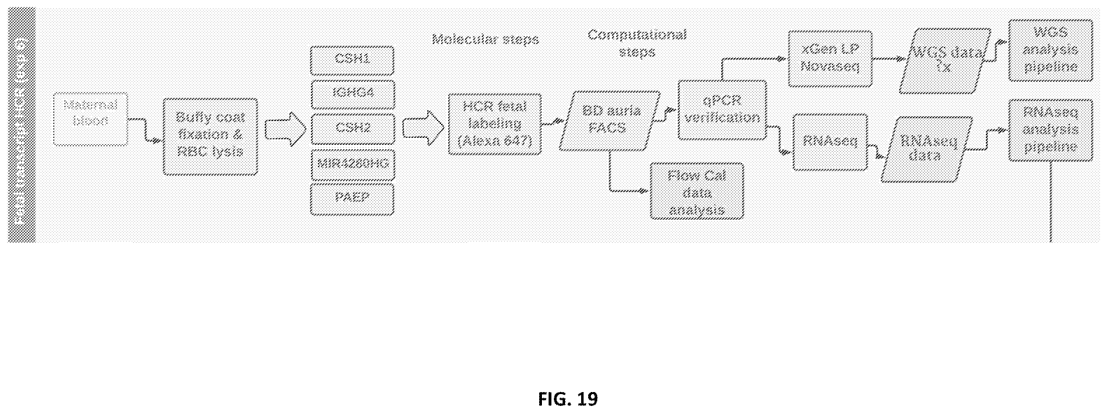

The present example demonstrates exemplary detection/isolation of putative CFCs using fetal placental-cell specific nucleic acid probes that specifically hybridize CSH-1/2 or pooled probe sets comprising a nucleic acid probe that specifically hybridizes with CSH-1/2-2. An exemplary workflow for detection/isolation of such cells and subsequent detection of the presence or absence of a genetic variant is shown in FIG. 19.

HCR probe sets targeting single fetal/placental cell-specific transcripts that specifically hybridized to CSH-1/2 were utilized in addition to pooled HCR probe sets which comprised HCR probe sets that targeted either of CSH1/2, IGHG4, and MIR4280HG. HCR probes further comprised a far-red detectable marker. Samples that went through the entirety of the HCR protocol, but in the absence of HCR probes, were utilized as a negative control.

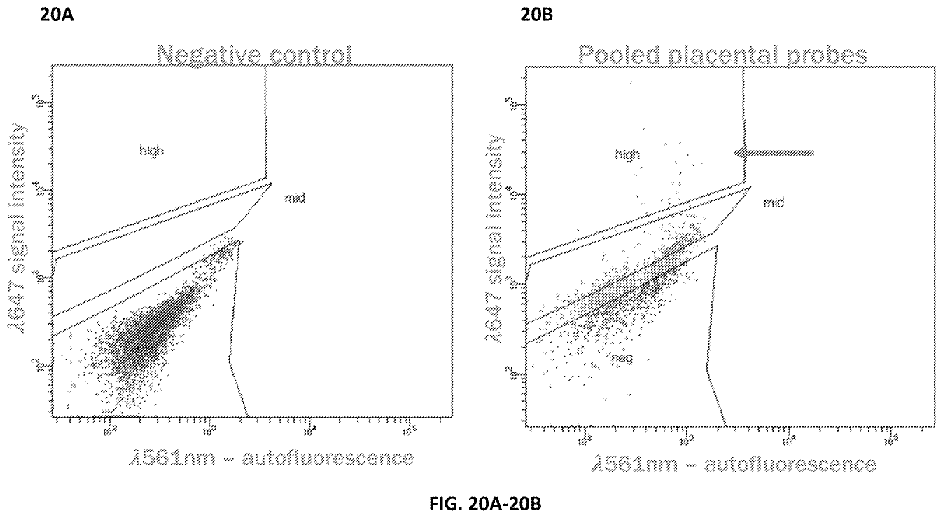

The HCR pooled probe set, including HCR probe sets that targeted CSH-1/2, IGHG4, and MIR4280HG, was added to the maternal blood sample to a final concentration of 64 nM. No putative CFCs were detected/isolated in the negative control group (FIG. 20A). The pooled HCR probe set detected/isolated 24 putative CFCs from the isolated buffy coat of maternal blood (FIG. 20B).

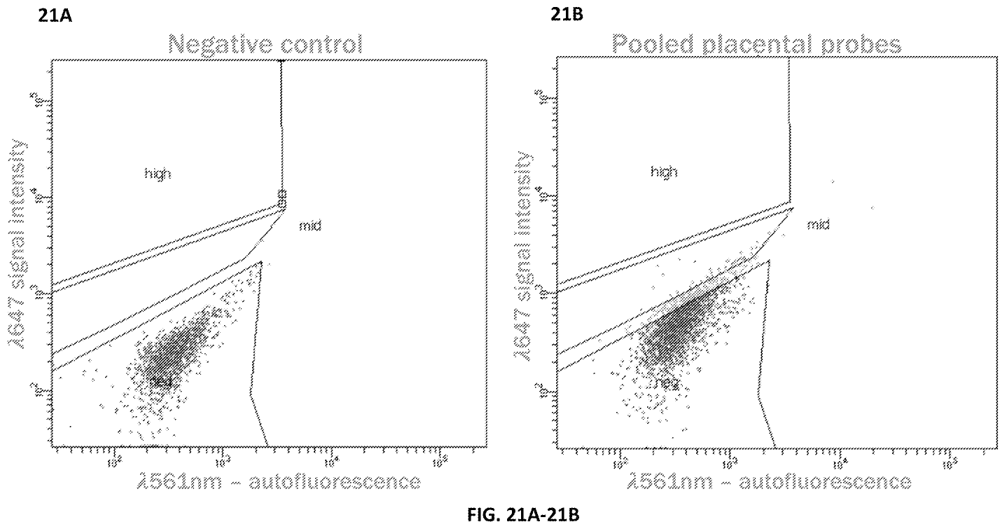

The HCR pooled probe set, including HCR probe sets that targeted CSH-1/2, IGHG4, and MIR4280HG, was also evaluated at a final concentration of 16 nM. No putative CFCs were detected/isolated in the negative control group (FIG. 21A). The pooled HCR probe set detected/isolated 24 putative CFCs from the isolated buffy coat of maternal blood (FIG. 21B).

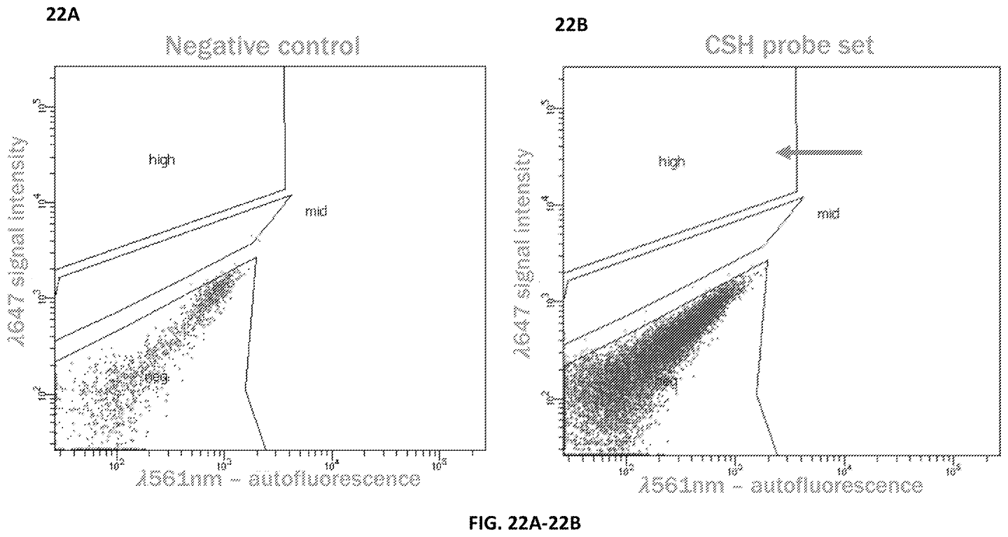

An HCR probe set that specifically hybridized to fetal/placental-specific transcript, CSH-1/2, was evaluated at a final concentration of 16 nM. No putative CFCs were detected/isolated in the negative control group (FIG. 22A). The CSH-1/2 only probe set did not detect/isolate any putative CFCs from the isolated buffy coat of maternal blood (FIG. 22B).

### Example 6: Exemplary Characterization of Isolated Cells

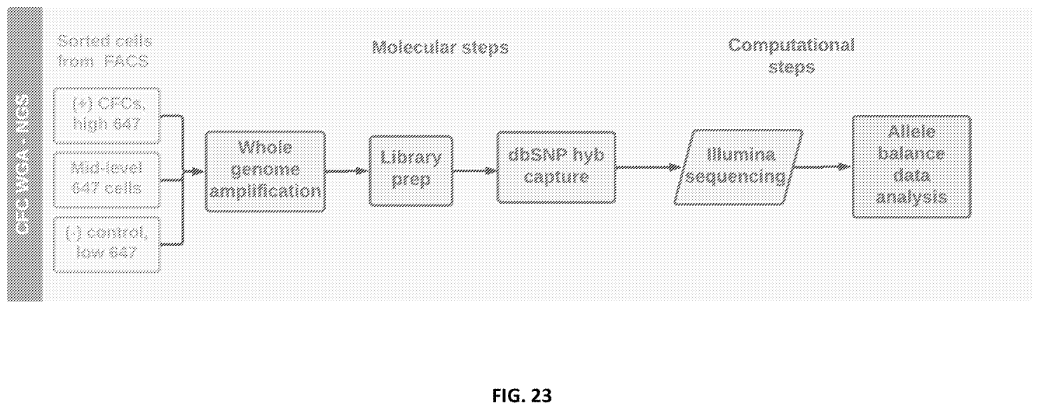

The present example demonstrates exemplary characterization of isolated cells. An exemplary workflow for characterization of isolated cells using whole genome amplification and Next Generation Sequencing (NGS) is shown in FIG. 23. Cells were isolated with high, medium, and low Alexa 647 fluorescence, corresponding to what is understood to be CFCs (high Alexa 647 fluorescence) and maternal cells (medium and low Alexa 647 fluorescence). Subsequently, whole genome amplification was conducted to increase the amount of genomic DNA for library preparation. A DNA library was prepared for whole genome sequencing using the Integrated DNA Technologies xGEn ffpe/cfDNA library preparation kit. Next, hybridization capture was completed to enrich for polymorphic regions of the genome that are frequently naturally occurring and comprise neutral (e.g., non-deleterious) SNPs. These allow for genotyping of the maternal verse CFCs.

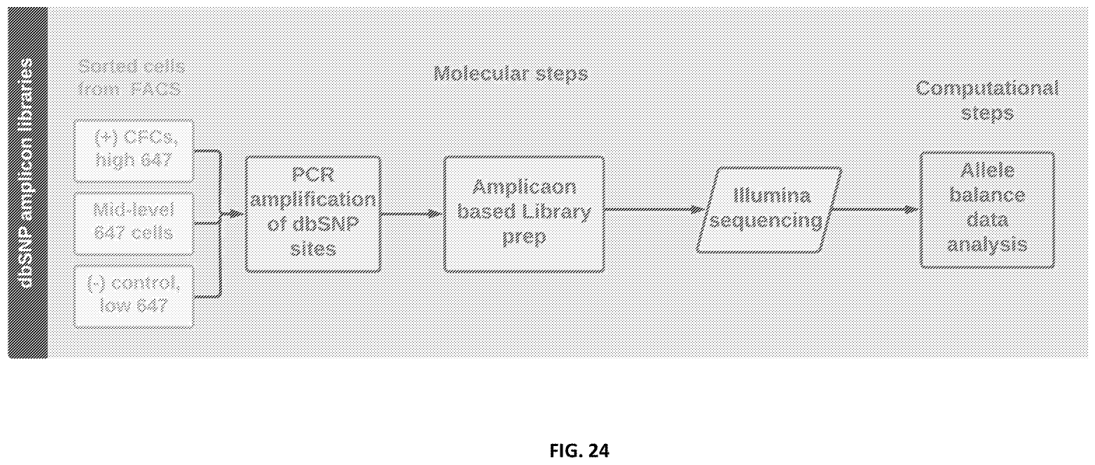

An exemplary workflow for characterization of isolated cells using direct amplification of dbSNP sites is shown in FIG. 24.

### Example 7: Exemplary Characterization of Allelic Balance

The present example demonstrates exemplary characterization of allelic balance in isolated cells. Allelic balance (AB) is a measure of the proportion of sequencing reads covering a variant's (e.g., SNPs) location that support the presence of the variant. A population of isolated cells, when characterized as in allelic balance (AB˜=0, AB˜=0.5, AB˜=1), is determined to be a pure population of isolated cells and/or as cells isolated from a single source.

Cells can be isolated and stained with fetal/placental-specific gene expression using FACS. Genomic DNA of isolated cell populations can be sequenced at high frequency SNP locations. High frequency SNPs observed were either homozygous for the reference, (AB=0), homozygous for the alternative allele (AB=1) or heterozygous (AB between 0.5 and 0.95). The genotype of fetal cells can be compared to that of maternal cells. Patterns are consistent with the fetal cells inheriting at one allele from the maternal cells and one allele from elsewhere (e.g., paternal inheritance), suggesting a pure population of fetal cells are isolated.

### Example 8: Exemplary Detection of Genetic Variants in Isolated Cells

The present example demonstrates detecting the presence or absence of genetic variants in isolated cells (e.g., rare cells). Cells, including rare cells, can be isolated according to methods described herein. A method of detecting the presence or absence of a genetic variant in a rare cell can include (a) contacting a sample with one or more nucleic acid probes comprising a nucleic acid sequence complementary to one or more cell-specific transcripts, wherein the nucleic acid probe comprises a detectable marker, thereby detectably labeling the cells; (b) separating the detectably labeled cells from one or more undesired sample components, thereby enriching the population of cells; (c) sequencing at least one nucleic acid from the enriched population of cells; and (d) detecting the presence or absence of the genetic variant based on the sequencing reads.

Sequencing at least one nucleic acid from the enriched population of cells can detect the presence of absence of genetic variants including, for example, aneuploidies (e.g., trisomy 21, trisomy 13, trisomy 18, 22q), pathogenic mutations in disease relevant genes (e.g., CFTR, HBB, p53), benign variants, loss-of-heterozygosity, inversions, translocations, chromothripsis, copy number changes (e.g., large copy-number alterations) in disease relevant genes (e.g., SMN1, HBA1, HBA2, RHD), and/or sex of a fetus.

1. A method of genotyping a rare cell comprising:
(a) contacting a sample with one or more nucleic acid probes comprising a nucleic acid sequence complementary to one or more rare cell-specific transcripts, wherein the nucleic acid probe comprises a detectable marker, thereby detectably labeling the rare cells;
(b) separating and collecting the detectably labeled rare cells from one or more undesired sample components, thereby isolating the rare cell; and
(c) genotyping the isolated rare cell.

2. The method of claim 1, wherein the rare cell is a circulating fetal cell.

3. The method of claim 2, wherein the one or more rare-cell specific transcripts is a fetal-cell-specific transcript.

4. The method of claim 2, further comprising diagnosing a fetus with a disease and/or disorder or determining that a fetus is at an increased risk of having a disease and/or disorder based on the presence or absence of a genetic variant in the circulating fetal cells.

5. The method of claim 1, wherein the rare cell is present in the sample at an abundance of about 1e⁻⁴% of the total number of cells in the sample.

6. (canceled)

7. (canceled)

8. The method of claim 1, wherein the one or more rare cell-specific transcripts are patient-specific.

9. The method of claim 1, wherein the one or more nucleic acid probes comprise hybridization chain reaction probes.

10. (canceled)

11. The method of claim 1, wherein the detectably labeled rare cells are separated from one or more undesired sample components using Fluorescence Activated Cell Sorting (FACS).

12. The method of claim 1, wherein the one or more rare cell-specific transcript comprises CSH1, IGHG4, CSH2, MIR4280HG, or any combination thereof.

13. The method of claim 1, further comprising verifying the genetic identity of the isolated rare cell.

14. The method of claim 1, further comprising sequencing at least one nucleic acid from the isolated rare cell.

15. The method of claim 1, wherein genotyping comprises one or more of:
karyotyping, polymerase chain reaction (PCR), short tandem repeat (STR) profiling, single nucleotide polymorphism (SNP) genotyping, DNA sequencing, RNA sequencing, use of cell type-specific nucleic acid probes, or any combination thereof.

16. A method of detecting the presence or absence of a genetic variant in a rare cell comprising:
(a) contacting a sample with one or more nucleic acid probes comprising a nucleic acid sequence complementary to one or more rare cell-specific transcripts, wherein the nucleic acid probe comprises a detectable marker, thereby detectably labeling a rare cell;
(b) separating the detectably labeled rare cell from one or more undesired sample components, thereby enriching the rare cell;
(c) sequencing at least one nucleic acid from the rare cell, to obtain sequence reads; and
(d) detecting the presence or absence of the genetic variant based on the sequencing reads.

17. The method of claim 16, wherein the rare cells comprise circulating fetal cells.

18. The method of claim 17, further comprising diagnosing a fetus with a disease and/or disorder or determining that a fetus is at an increased risk of having a disease and/or disorder based on the presence or absence of a genetic variant in the circulating fetal cells.

19. The method of claim 16, wherein the rare cells are present in the sample at an abundance of about 1e⁻⁴% of the total number of cells in the sample.

20. (canceled)

21. (canceled)

22. The method of claim 16, wherein the one or more nucleic acid probes comprise hybridization chain reaction probes.

23. The method of claim 16, wherein the detectable marker is or comprises a fluorescent molecule.

24. The method of claim 16, wherein the detectably labeled rare cells are separated from one or more undesirable sample components using Fluorescence Activated Cell Sorting (FACS).

25. The method of claim 16, wherein sequencing at least one nucleic acid from the enriched population of rare cells comprises next generation sequencing.
